(19) United States
(12) Patent Application Publication
(10) Pub. No.: US 2025/0186432 A1
(43) Pub. Date: Jun. 12, 2025

(54) SPLEEN TYROSINE KINASE INHIBITOR, COMPOSITION, AND METHODS OF USE

(71) Applicant: PURDUE RESEARCH FOUNDATION, West Lafayette, IN (US)

(72) Inventors: Philip Stewart Low, West Lafayette, IN (US); Madduri Srinivasarao, West Lafayette, IN (US); John Michael Hausman, San Mateo, CA (US); Suresh Yarlagadda, West Lafayette, IN (US)

(21) Appl. No.: 18/847,164

(22) PCT Filed: Mar. 14, 2023
(86) PCT No.: PCT/US2023/064326

§ 371 Date: Sep. 13, 2024

Related U.S. Application Data
(60) Provisional application No. 63/398,599, filed on Aug. 17, 2022.
(60) Provisional application No. 63/319,486, filed on Mar. 14, 2022.

(51) Int. Cl. A61K 31/4985; A61K 31/05; A61K 31/17; A61K 31/404; A61K 31/437; A61K 31/497; A61K 31/501; A61K 31/505; A61K 31/506; A61K 31/519; A61K 31/53; A61K 31/5377; A61K 31/675; C07D 403/04; C07D 403/14; C07D 487/04; C07F 9/6558

(52) U.S. Cl. CPC A61K 31/4985; A61K 31/05; A61K 31/17; A61K 31/404; A61K 31/437; A61K 31/497; A61K 31/501; A61K 31/505; A61K 31/506; A61K 31/519; A61K 31/53; A61K 31/5377; A61K 31/675; C07D 403/04; C07D 403/14; C07D 487/04; C07F 9/65583

(57) ABSTRACT

Compounds, compositions and methods for inhibiting Syk from phosphorylating erythrocyte anion transporter band 3 and/or Syk tyrosine kinase. Further disclosed are methods of treating a subject for a disease involving the release of microvesicles from blood cells, including alpha-thalassemia or beta-thalassemia, sickle cell disease, glucose-6-phosphate dehydrogenase (G6PD) deficiency or glutathione reductase deficiency.

### PRIORITY

This patent application is related to and claims the priority benefit of U.S. Provisional Patent Application No. 63/319,486 filed Mar. 14, 2022, and U.S. Provisional Patent Application No. 63/398,599 filed Aug. 17, 2022. The contents of the foregoing applications are hereby incorporated by reference in their entireties into this disclosure.

### STATEMENT OF GOVERNMENT SUPPORT

This invention was made with government support under GM024417 awarded by the National Institutes of Health. The government has certain rights in the invention.

### TECHNICAL FIELD

The present disclosure relates to a spleen tyrosine kinase (Syk) inhibitor, a composition comprising the same, and methods of use in the treatment of sickle cell disease and other diseases involves microvesicle release from blood cells.

### BACKGROUND

Sickle cell disease is a group of hereditary blood disorders due to an abnormality of hemoglobin (specifically an abnormality in the structure of β-globin (glu6val)), the molecule in red blood cells (RBCs) that delivers oxygen to cells throughout the body. Sickle cell can be characterized by the mutation of an amino acid of this βchain from glutamic acid to valine, which causes deoxygenated sickle hemoglobin (deoxy-IbS) to form insoluble polymers inside RBCs and deforms the RBCs into rigid shapes or sickle (i.e., crescent) cells.

Sickle cell disease affects a significant number of individuals worldwide. In the United States, those with sickle cell disease can have an average mortality in their 40 s, a poor quality of life, and high medical costs. Signs and symptoms of sickle cell disease usually begin in early childhood. Characteristic features of this disorder can include a low number of RBCs (i.e., anemia), repeated infections, and periodic episodes of pain.

The signs and symptoms of sickle cell disease are caused by the sickling of RBCs. When RBCs sickle, they can break down prematurely which can lead to anemia and other complications such as jaundice, pulmonary hypertension, and heart failure.

Currently, the standard of care for sickle cell patients two years of age and older is blood transfusions and treatment with hydroxyurea, which is an anti-cancer drug. Not all patients respond to hydroxyurea, and it can be poorly tolerated causing myelosuppression in some. A pyruvate kinase agonist-based treatment is also available (e.g., imatinib); however, such inhibitors can also interact with (i.e., inhibit) growth factor receptors involved in the revascularization of tissues which is often needed due to the common occurrence of vaso-occlusive events in sickle cell patients. Additionally, unwanted inhibition of growth factor receptors can be especially problematic in children as it can interfere with their growth and development.

In view of the above, it is an object of the present disclosure to provide such a therapeutic agent. This and other object and advantages, as well as inventive features, will be apparent from the detailed description provided herein.

### SUMMARY

Provided is a compound:
(i) which comprises a rigid, fused bicyclic ring, which is substituted with at least two substituents, at least one of which is an N-linked, optionally substituted, carbo- or heterocycle and the other of which is an indole substituted with at least one substituent, at least one of which is a 4-6-membered, optionally substituted, carbo- or heterocycle,
(ii) which inhibits spleen tyrosine kinase (Syk) from phosphorylating the human erythrocyte anion transporter band 3, and
(iii) which inhibits Syk tyrosine kinase at a concentration that is at least about 20-fold lower than the concentration at which the compound inhibits hepatocyte growth factor receptor (HGFR), epidermal growth factor receptor (EGFR), fibroblast growth factor receptor 3 (FGFR3), insulin-like growth factor 1 receptor (IGF1R), and/or a vascular endothelial growth factor receptor (VEGFR),
wherein, additionally or alternatively to (ii) and (iii), the compound of (i) has:
(iv) a CLogP value between about 2 and about 5 (e.g., between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds.

The compound can be (e.g., have the structure of) formula X:

A−B−C (X)

or be a pharmaceutically acceptable salt thereof, wherein:
A is a first ring that is nitrogen-linked and optionally substituted, the first ring comprising a fused bicyclic ring or a monocyclic ring with each ring each first ring comprising a 4-6-membered heterocycle or carbocycle;
B is a second ring, optionally substituted with at least one substituent, the second ring comprising a monocyclic ring or a fused bicyclic or tricyclic ring, and wherein each ring of the second ring comprises a 5- or 6-membered heterocycle or carbocycle; and
C is a third bicyclic ring comprising a 5- or 6-membered carbocycle fused with a 5- or 6-membered carbocycle or heterocycle, wherein the third bicyclic ring is substituted with at least a first substituent comprising:

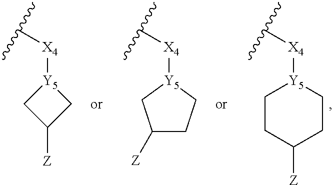

or an amine, or an alcohol,
wherein:
   X₄ is CH₂, NH, CO, O, or S,
   Y₅ is CH or N,
   Z is selected from OH, SH, NH₂, CO₂H, NHCH₃, and

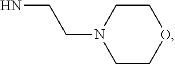

and [symbol] is a point of attachment of the first substituent to the third bicyclic ring.

In certain embodiments, A of the compound is:

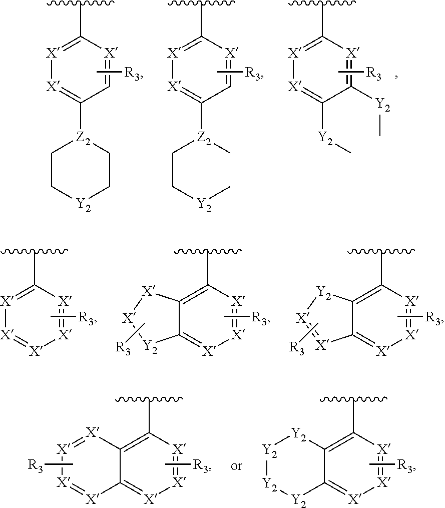

wherein:
each X′ is independently selected from CH and NH;
each Y₂ is independently selected from CH₂, NH, O, and S;
Z₂ is CH or N; and
each R₃ is independently selected from H, F, Cl, OH, and —Y—(CH₂)ₙ—, in which n=1-14.

The N-linked to the first ring of A of the compound can form a nitrogen bridge with B of the compound. In certain embodiments, A is a first monocyclic ring substituted with a bulky substituent para to such nitrogen bridge. The bulky substituent can be morpholine, for example.

In certain embodiments, each ring of the first ring of A is a 6-membered, optionally substituted, open or closed, heterocycle or carbocycle. A can be a first monocyclic ring substituted with two methoxy groups.

In certain embodiments, B of the compound is:

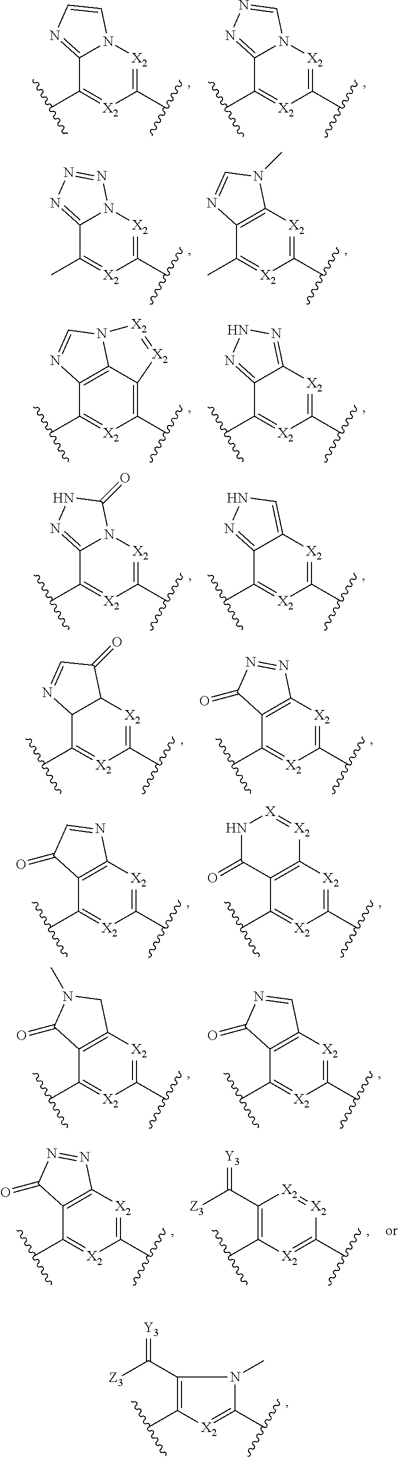

wherein:
each X₂ is independently selected from CH and N,
Y₃ is selected from O, NH and S; and
Z₃ is selected from OH, NH₂, and NHCH₃.

In certain embodiments, B of the compound is not

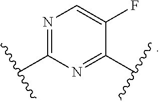 [SMILES: *c1ncc(F)c(*)n1]

In other words, the compound is any of those described herein, with the proviso that B is not

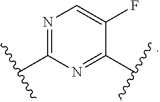 [SMILES: *c1ncc(F)c(*)n1]

B can be a monocyclic ring substituted with an amide. B can be rigid. In certain embodiments, B is a pyrazine substituted with at least one substituent (e.g., an amide).

C of the compound can be an indole. C of the compound can be:

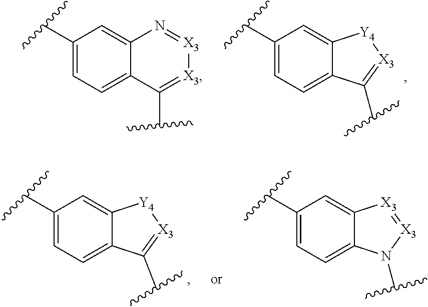

wherein:
each X₃ is independently selected from CH and N; and
Y₄ is CH₂, NH, O, or SH.

The third bicyclic ring of C can be an indo and the first substituent of C can comprise a 4-6-membered, optionally substituted, carbo- or heterocycle substituent. In certain embodiments, the carbo- or heterocycle substituent is a lipophilic ester.

The first substituent of C can be

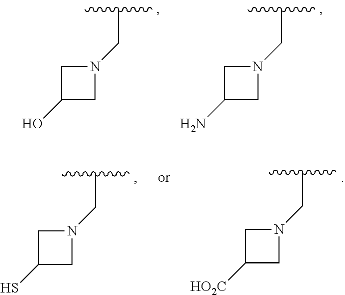 [SMILES: *CN1CC(O)C1 ; *CN1CC(N)C1 ; *CN1CC(S)C1 ; *CN1CC(C(=O)O)C1]

The Z of the first substituent of X can be —OR in which R is

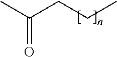

and n=1-14.

In certain embodiments, Z of the first substituent of the third bicyclic ring comprises a phosphate or phosphate ester. In certain embodiments, the first substituent of C comprises an —OR, wherein the R is a phosphate or phosphonate ester. In certain embodiments, the first substituent of C is

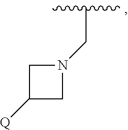

wherein Q is a phosphate or phosphonate ester. In certain embodiments, the first substituent of C is

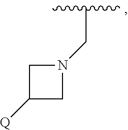

wherein Q is RC(═O)—, in which R is an alkyl (e.g., a C₁-C₆ alkyl).

B of the compound can form a hydrogen bond with amino acid residue Ala451 in a catalytic domain of Syk tyrosine kinase and the first substituent of C (e.g., Z thereof) can form a hydrogen bond with amino acid residue Aspe2 in a catalytic domain of Syk tyrosine kinase.

One or more of the hydrogens of the compound can be optionally substituted with deuterium.

The compound can comprise the structure:

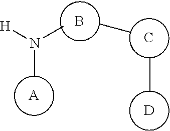

in which ring A is

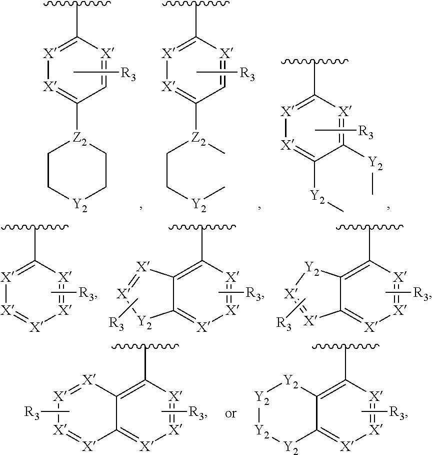

wherein:
   each X′ is independently selected from CH and NH;
   each Y is independently selected from CH₂, NH, O, and S;
   Z₂ is CH or N; and
      each R₃ is independently selected from H, F, Cl, OH, and —Y—(CH₂)ₙ—, in
which n=1-14;
in which ring B is

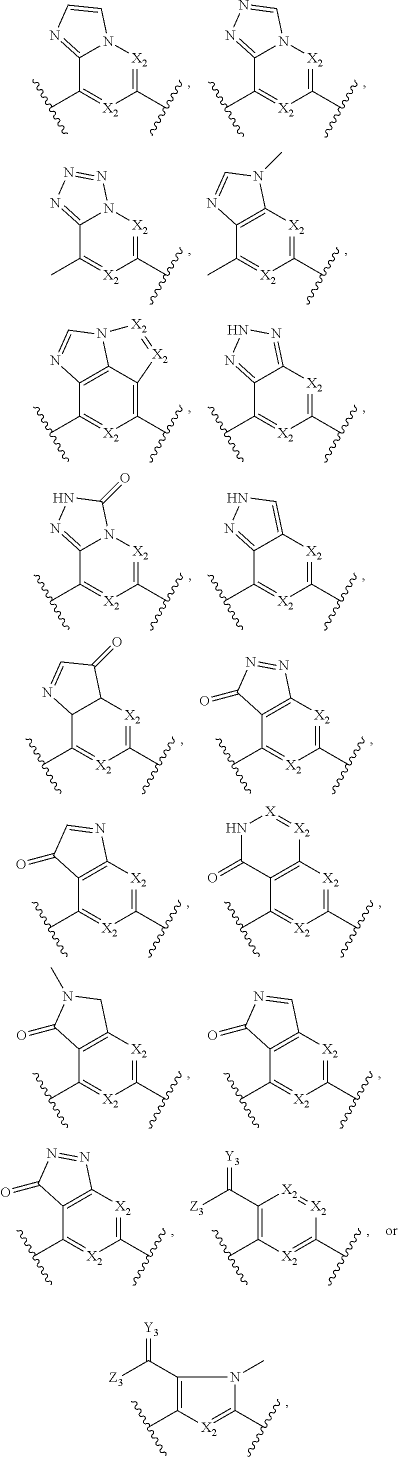

where in:
   each X₂ is independently selected from CH and N;
   Y_{z} is selected from 0, NIH and S; and
   Z₃ is selected from OH, NH₂, and NHCH₃;
with the proviso that B is not

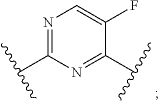 [SMILES: *c1ncc(F)c(*)n1]

in which ring C is

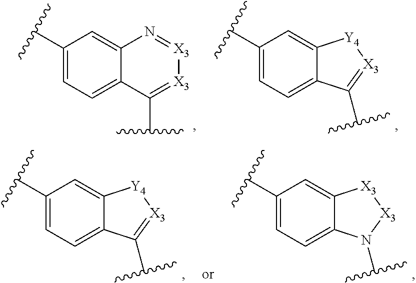

wherein:
   each X₃ is independently selected from CH and N; and
   Y₄ is selected from CH₂, NH, O, and SH;
in which ring D is

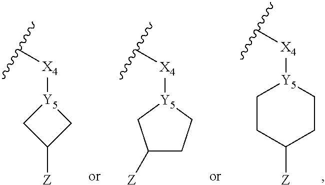

or an amine, or an alcohol,
wherein:
   X₄ is selected from CH₂, NH, CO, O, and S;
   Y₅ is selected from CH and N; and
   Z is selected from OH, SH, NH₂, NHCH₃, and

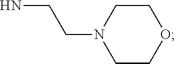

and
one or more hydrogens can be optionally substituted with deuterium.

The ring B can form a hydrogen bond with the amino acid residue Ala451 in the catalytic domain of Syk tyrosine kinase and Z on ring D (e.g., a first substituent of C) can form a hydrogen bond with amino acid residue Asp512 in the catalytic domain of Syk tyrosine kinase. The ring A can be substituted with a bulky substituent para to the nitrogen bridge. The bulky substituent can be a 6-membered, optionally substituted, open or closed, carbo- or heterocycle. The bulky substituent can be morpholine.

The compound can have an increased docking score for Syk tyrosine kinase as compared to an otherwise identical compound lacking the bulky substituent para to the nitrogen bridge. The compound can comprise a structure of:

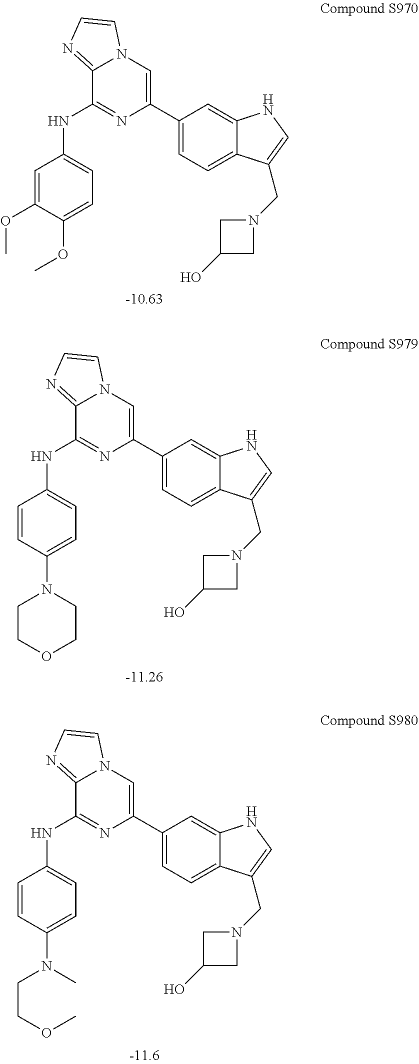 [SMILES: COc1ccc(Nc2nc(-c3ccc4c(CN5CC(O)C5)c[nH]c4c3)cn3ccnc23)cc1OC ; OC1CN(Cc2c[nH]c3cc(-c4cn5ccnc5c(Nc5ccc(N6CCOCC6)cc5)n4)ccc23)C1 ; COCCN(C)c1ccc(Nc2nc(-c3ccc4c(CN5CC(O)C5)c[nH]c4c3)cn3ccnc23)cc1]

The compound can comprise or have the structure:

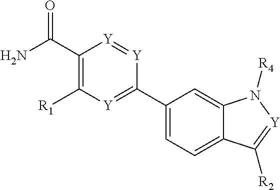

wherein:

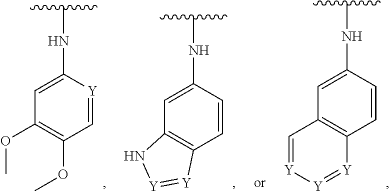

R₁ is
R₂ comprises

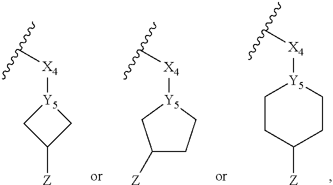

or an amine, or an alcohol, wherein:
   X₄ is CH NH, CO, O, or S,
   Y₅ is CH or N,
   Z is selected from OH, SH, NH₂, CO₂H, NHCH₃, and

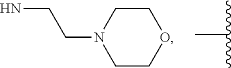

is a point of attachment of the first substituent to the third bicyclic ring; and
R₄ is H, CH₃, an amine, or a methanol.

The compound can comprise the structure:

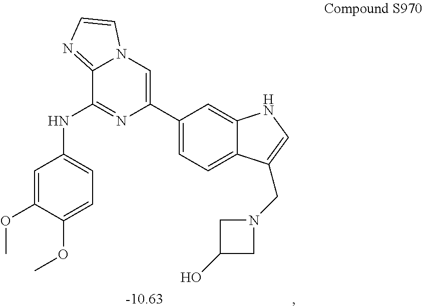 [SMILES: COc1ccc(Nc2nc(-c3ccc4c(CN5CC(O)C5)c[nH]c4c3)cn3ccnc23)cc1OC]

or be a pharmaceutically acceptable salt thereof.

The compound can comprise the structure:

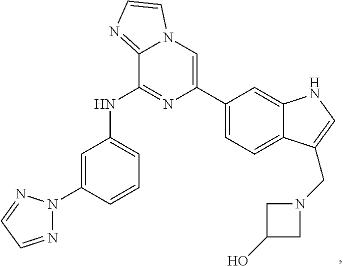 [SMILES: OC1CN(Cc2c[nH]c3cc(-c4cn5ccnc5c(Nc5cccc(-n6nccn6)c5)n4)ccc23)C1]

or be a pharmaceutically acceptable salt thereof.

The compound can comprise the structure:

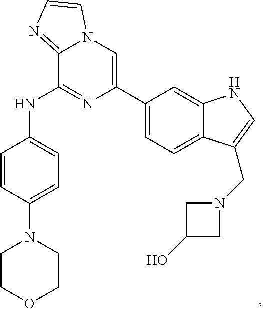 [SMILES: OC1CN(Cc2c[nH]c3cc(-c4cn5ccnc5c(Nc5ccc(N6CCOCC6)cc5)n4)ccc23)C1]

or be a pharmaceutically acceptable salt thereof.

The compound can have or comprise the structure:

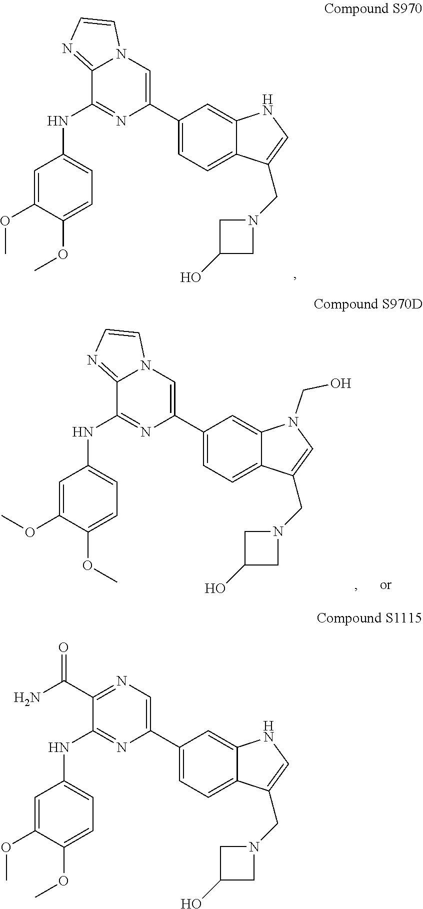 [SMILES: COc1ccc(Nc2nc(-c3ccc4c(CN5CC(O)C5)c[nH]c4c3)cn3ccnc23)cc1OC ; COc1ccc(Nc2nc(-c3ccc4c(CN5CC(O)C5)cn(CO)c4c3)cn3ccnc23)cc1OC ; COc1ccc(Nc2nc(-c3ccc4c(CN5CC(O)C5)c[nH]c4c3)cnc2C(N)=O)cc1OC]

or be a pharmaceutically acceptable salt of any of the foregoing.

The compound can comprise the structure:

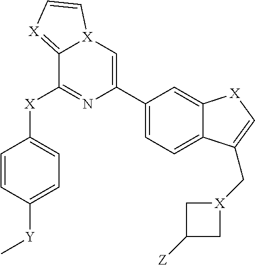

or be a pharmaceutically acceptable salt thereof,
in which each X is independently selected from CH₂, N, NH, O, and S;
Y is O, NH, or SH; and
Z is OH or NH₂. The 4-6-membered, optionally substituted, carbo- or heterocycle substituent on the indole can comprise a lipophilic ester. The Z on ring D can be —OR in which R is

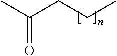

and n=1-14.

The compound can have or comprise, or be a pharmaceutically acceptable salt of the structure:

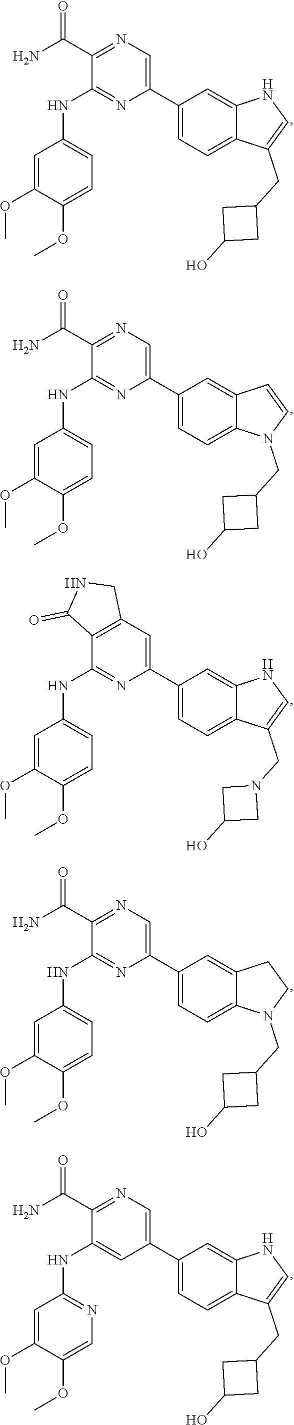

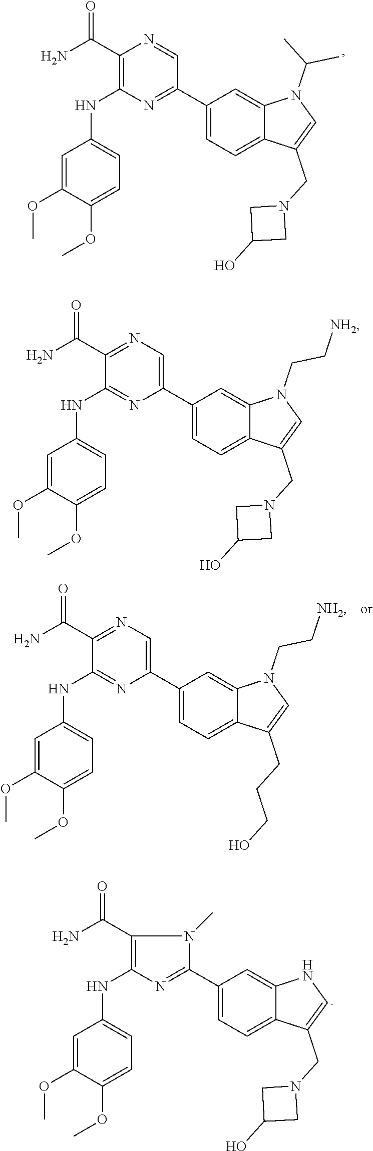

In certain embodiments, the compound has or comprises, or is a pharmaceutically acceptable salt of, the structure:

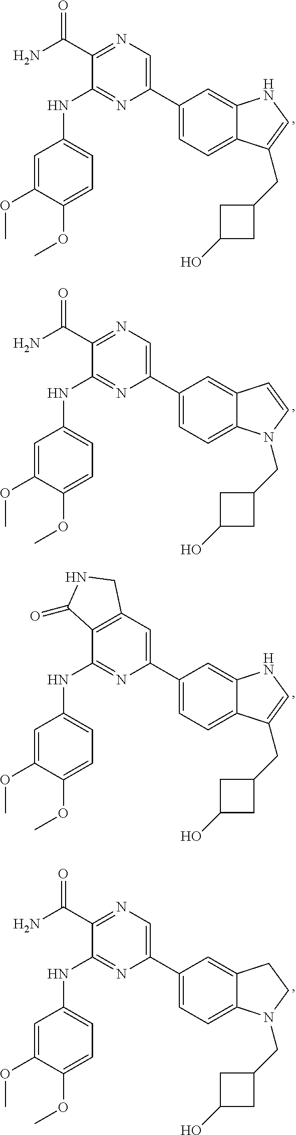 [SMILES: COc1ccc(Nc2nc(-c3ccc4c(CC5CC(O)C5)c[nH]c4c3)cnc2C(N)=O)cc1OC ; COc1ccc(Nc2nc(-c3ccc4c(ccn4CC4CC(O)C4)c3)cnc2C(N)=O)cc1OC ; COc1ccc(Nc2nc(-c3ccc4c(CC5CC(O)C5)c[nH]c4c3)cc3c2C(=O)NC3)cc1OC ; COc1ccc(Nc2nc(-c3ccc4c(c3)CCN4CC3CC(O)C3)cnc2C(N)=O)cc1OC]

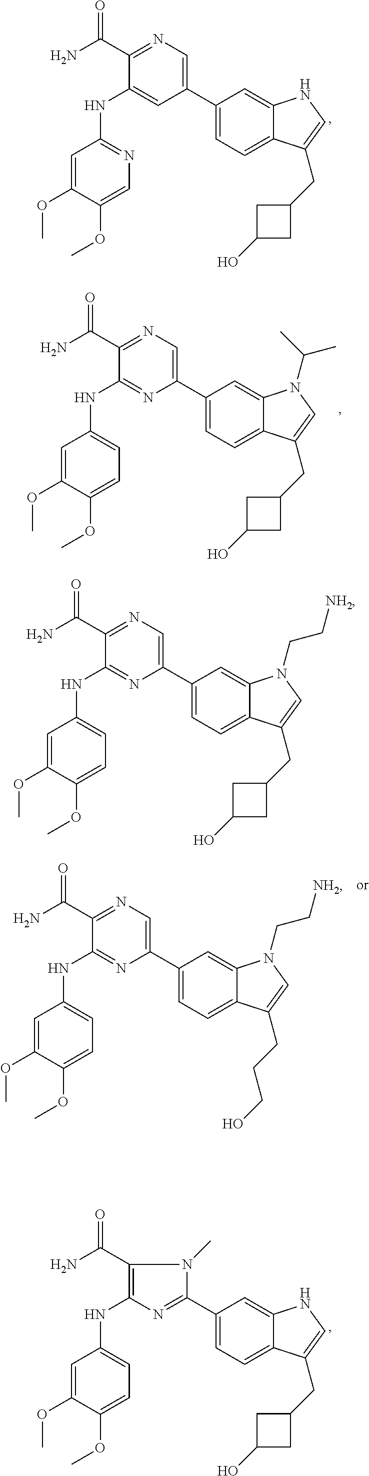

wherein the hydroxyl (HO—) group is replaced with: (a) a phosphate or a phosphonate ester; or (b) RC(═O)O—, in which R is an alkyl (e.g., C₁-C₆ alkyl).

The compound can be a compound:
(i) comprising a pyrazine, which is substituted with at least three substituents, at least one of which is a carboxamide or a lactam, another of which is an N-linked, optionally substituted, carbo- or heterocycle, and another of which is a carbo- or heterocycle with at least one substituent comprising a 4-6-membered, optionally substituted, carbo- or heterocycle,
(ii) which inhibits Syk tyrosine kinase from phosphorylating the human erythrocyte anion transporter band 3,
(iii) which inhibits Syk tyrosine kinase at a concentration that is at least about 20-fold lower than the concentration at which the compound inhibits HGFR, EGFR), FGFR3, IGF1R, and/or a VEGFR,
wherein, additionally or alternatively to (ii) and (iii), the compound of (i) has:
(iv) a clogP value between about 2 and about 5 (e.g., between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds.

The compound can have or comprise, or be a pharmaceutically acceptable salt of, the structure:

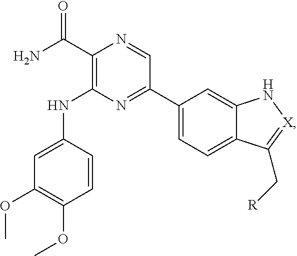

wherein X is CH or N, and R is

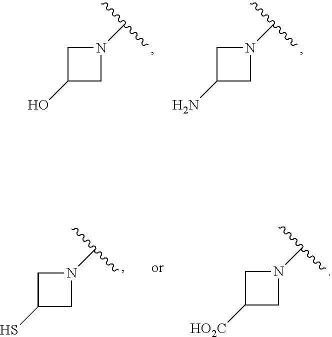 [SMILES: *N1CC(O)C1 ; *N1CC(N)C1 ; *N1CC(S)C1 ; *N1CC(C(=O)O)C1]

The compound can have or comprise, or be a pharmaceutically acceptable salt of, the structure:

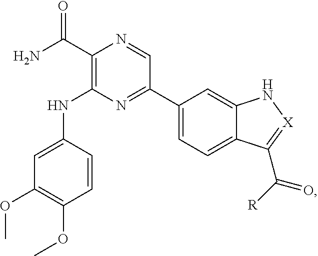

wherein X is CH or N, and R is

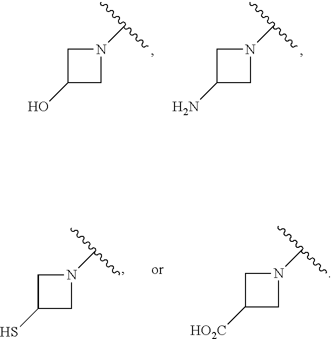 [SMILES: *N1CC(O)C1 ; *N1CC(N)C1 ; *N1CC(S)C1 ; *N1CC(C(=O)O)C1]

The compound can have or comprise, or be a pharmaceutically acceptable salt of, the structure:

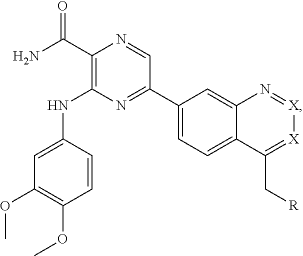

wherein X is CH or N, and R is

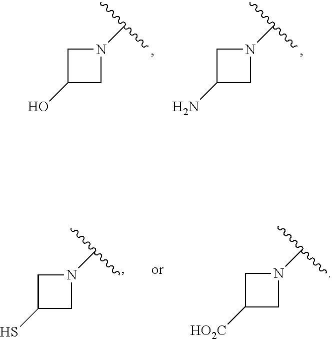 [SMILES: *N1CC(O)C1 ; *N1CC(N)C1 ; *N1CC(S)C1 ; *N1CC(C(=O)O)C1]

The compound can have or comprise, or be a pharmaceutically acceptable salt of, the structure:

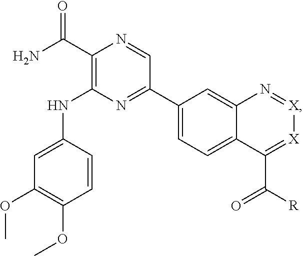

wherein X is CH or N, and R is

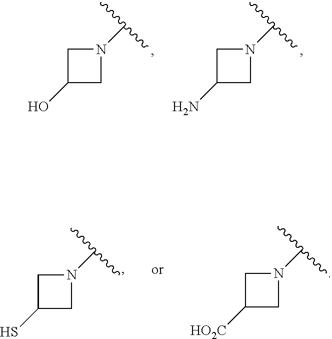 [SMILES: *N1CC(O)C1 ; *N1CC(N)C1 ; *N1CC(S)C1 ; *N1CC(C(=O)O)C1]

The compound can have or comprise, or be a pharmaceutically acceptable salt of, the structure:

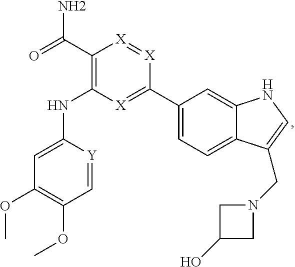

wherein X is CH or N; Y is CH or N, and when X and Y are all CH, the compound can have weaker binding affinity as compared to when at least one or more X and/or Y are N.

The compound can have or comprise, or be a pharmaceutically acceptable salt of, the structure:

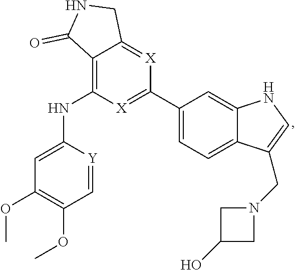

wherein X is CH or N; Y is CH or N, and when X and Y are all CH, the compound can have weaker binding affinity as compared to when at least one or more X and/or Y are N.

The compound can have or comprise, or be a pharmaceutically acceptable salt of, the structure:

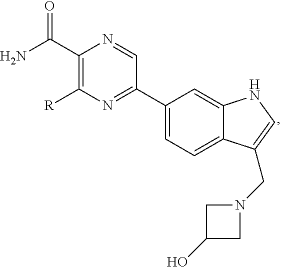

wherein R is

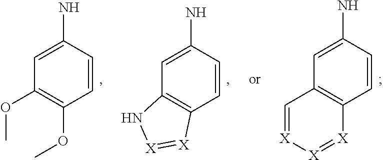

and each X is independently CH or N.

The compound can have or comprise, or be a pharmaceutically acceptable salt of, the structure:

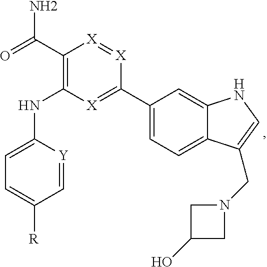

wherein each X is independently CH or N; each Y is CH or N; and R is carbo- or heterocyclic; and wherein when X and Y are all CH, the compound can have weaker binding affinity as compared to when at least one or more X and/or Y are N.

The compound can have or comprise, or be a pharmaceutically acceptable salt of, the structure:

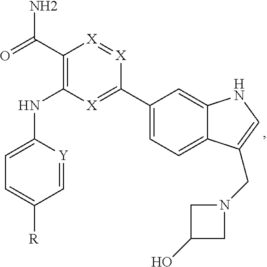

wherein each X is independently CH or N; Y is CH or N; and R is carbo- or heterocyclic; and wherein when X and Y are all CH, the compound can have weaker binding affinity as compared to when at least one or more X and/or Y are N.

The compound can have or comprise, or be a pharmaceutically acceptable salt of, the structure:

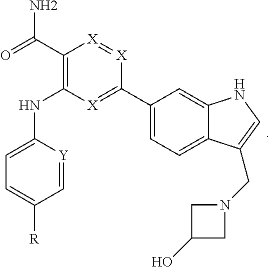

wherein each X is independently CH or N; Y is CH or N; and R is carbo- or heterocyclic; and wherein when X and Y are all CH, the compound can have weaker binding affinity as compared to when at least one or more X and/or Y are N.

The compound can have or comprise, or be a pharmaceutically acceptable salt of, the structure:

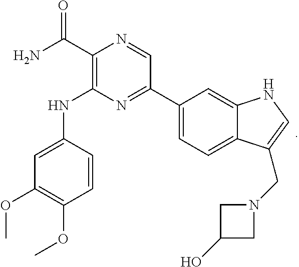 [SMILES: COc1ccc(Nc2nc(-c3ccc4c(CN5CC(O)C5)c[nH]c4c3)cnc2C(N)=O)cc1OC]

In an alternative embodiment, the hydroxyl (HO—) group of the foregoing compound can be replaced with phosphate or a phosphonate ester. In yet another alternative embodiment, the hydroxyl (HO—) group can be replaced with RC(═O)O—, in which R is an alkyl (e.g., a C₁-C₆ alkyl).

Further provided is a pharmaceutical composition comprising compound (or pharmaceutical salt thereof) described herein, and a pharmaceutically acceptable carrier or excipient. The composition can comprise cremophor, polysorbate, nanoparticles, a polymer, or a hydrogel, for example.

Still further provided is a method of treating sickle cell disease. The method comprises administering an effective amount of: (a) an above-described compound; (b) fostamatinib; (c) PRT062607; (d) TAK-659; (e) TAE-684; (f) entospleinib; (g) lanraplenib; (h) cerdulatinib; (i) piceatannol; (j) S701; (k) Syk II; (l) Syk IV; (m) TAS05567; (n) GSK143; (o) Syk-IN-3; (p) Syk-IN-4; (q) Syk-IN-1; (r) SRX3207; (s) RO9021; (t) gusacitinib; (u) R112; (v) PRT-060318; (w) OXSI-2; (x) (a) and (b); (y) (a) and (c); (z) (a) and (d); (aa) (a) and (e); (ab) (a) and (f); (ac) (a) and (g); (ad) (a) and (h); (ae) (a) and (i); (at) (a) and (j); (ag) (a) and (k); (ah) (a) and (l); (ai) (a) and (m); (aj) (a) and (n); (ak) (a) and (o); (al) (a) and (p); (am) (a) and (q); (an) (a) and (r); (ao) (a) and (s); (ap) (a) and (t); (aq) (a) and (u); (ar) (a) and (v); (as) (a) and (w); or (at) (a) and one or more of (b)-(w); optionally as a pharmaceutical composition comprising a pharmaceutically acceptable carrier or excipient.

Methods for treating a red blood cell (RBC)-mediated disease are also provided. In certain embodiments, a method for treating an RBC-mediated disease comprises administering to a subject (e.g., a human patient) an effective amount of: a compound as described herein or pharmaceutically acceptable salt thereof, or a pharmaceutical composition described herein. In certain embodiments, the compound is:
a compound of formula X:

A-B-C (X)

or a pharmaceutically acceptable salt thereof wherein:
A is a first ring that is nitrogen-linked and optionally substituted, the first ring comprising a fused bicyclic ring or a monocyclic ring with each ring each first ring comprising a 4-6-membered heterocycle or carbocycle;
B is a second ring, optionally substituted with at least one substituent, the second ring comprising a monocyclic ring or a fused bicyclic or tricyclic ring, and wherein each ring of the second ring comprises a 5- or 6-membered heterocycle or carbocycle; and
C is a third bicyclic ring comprising a 5- or 6-membered carbocycle fused with a 5- or 6-membered carbocycle or heterocycle, wherein the third bicyclic ring is substituted with at least a first substituent comprising:

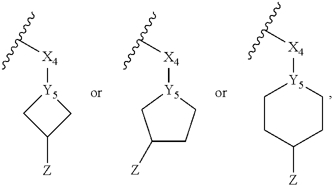

or an amine, or an alcohol,
wherein:
   X₄ is each independently CH₂, NH, CO, 0, or S,
   Y₅ is each independently CH or N,
   Z is selected from OH, SH, NH₂, CO₂H, NHCH₃, and

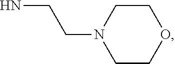

and [symbol] is a point of attachment of the first substituent to the third bicyclic ring,
wherein the compound inhibits Syk tyrosine kinase at a concentration that is at least 20-fold lower than the concentration at which the compound inhibits HGFR, EGFR, FGFR3, IGF1R, and/or a VEGFR; or
is a pharmaceutical composition comprising the above-described compound or a pharmaceutically acceptable salt thereof.

The RBC-mediated disease can be selected from the group consisting of sickle cell disease, thalassemia, glucose-6-phosphate dehydrogenase deficiency, glutathione reductase deficiency, and a disease involving the release of microvesicles from blood cells. In certain embodiments, the RBC-mediated disease is α-thalassemia or β-thalassemia, alone or in combination with sickle cell disease.

A method of preventing or inhibiting the phosphorylation of a human erythrocyte anion transporter band 3 in a subject is also provided. In certain embodiments, the method comprises administering to the subject an effective amount of a compound (or pharmaceutically acceptable salt thereof) described herein, or a pharmaceutical composition hereof.

In each method hereof, administration of the compound, pharmaceutical salt thereof, or pharmaceutical composition to the subject can stabilize erythrocyte cell membranes in the subject and reduce the release of hemoglobin from sickle cells in the subject. In this manner, for example, the risk of vaso-occlusive crisis in the subject can be reduced. In certain embodiments, the compound, pharmaceutical salt thereof, or pharmaceutical composition inhibits Syk tyrosine kinase at an ICs of 200 nM or lower. In certain embodiments, the subject is a human child.

In certain embodiments, the methods hereof further comprise administering to the subject an effective amount of at least one second active agent. The at least one second active agent can be selected from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, Syk-IN-1, SRX3207, RO9021, gusacitinib, R112, PRT-060318, and OXSI-2. In certain embodiments, the at least one second active agent is two or more of ostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, Syk-IN-1, SRX3207, R09021, gusacitinib, R112, PRT-060318, and OXSI-2. The at least one second active agent can be administered with the compound (or a pharmaceutically acceptable salt thereof) or pharmaceutical composition simultaneously or sequentially, in either order.

The method can further comprise administering to the subject an effective amount of hydroxyurea. The hydroxyurea can be administered with the compound (or a pharmaceutically acceptable salt thereof) or pharmaceutical composition simultaneously or sequentially, in either order.

In certain embodiments, the at least one second active agent comprises more than one second active agent. In certain embodiments, the more than one second active agents are all selected from the from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, SRX3207, R09021, gusacitinib, R112, PRT-060318, and OXSI-2, optionally wherein the at least one second active agent is formulated as a pharmaceutical composition comprising a pharmaceutically acceptable carrier or excipient.

Another method of treating sickle cell disease in a subject is also provided. The method comprises administering an effective amount of a compound or a pharmaceutically acceptable salt thereof:
(i) which comprises a rigid, fused bicyclic ring, which is substituted with at least two substituents, at least one of which is an N-linked, optionally substituted, carbo- or heterocycle and the other of which is an indole substituted with at least one substituent, at least one of which is a 4-6-membered, optionally substituted, carbo- or heterocycle,
(ii) which inhibits Syk from phosphorylating the human erythrocyte anion transporter band 3,
(iii) which inhibits Syk tyrosine kinase at a concentration that is at least about 20-fold lower than the concentration at which the compound inhibits HGFR, EGFR, FGFR3, IGF1R, and/or a VEGFR,
wherein, additionally or alternatively to (ii) and (iii), the compound of (i) has:
(iv) a CLogP value between about 2 and about 5 (e.g., between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds, where upon the subject is treated for sickle cell disease. The compound (or a pharmaceutically acceptable salt thereof) can inhibit Syk from phosphorylating tyrosine 8 and tyrosine 21 in the human erythrocyte anion transporter band 3. The compound (or a pharmaceutically acceptable salt thereof) can stabilize erythrocyte cell membranes and reduce the release of microvesicles from sickle cells. The compound (or a pharmaceutically acceptable salt thereof) can stabilize erythrocyte cell membranes and reduce the release of hemoglobin from sickle cells. The risk of vaso-occlusive crisis in the subject can be reduced. The compound (or a pharmaceutically acceptable salt thereof) can inhibit Syk tyrosine kinase at an IC₅₀ of 200 nM or lower. The subject can be a human child.

The method can further comprise the simultaneous or sequential administration, in either order, of (a) the compound, pharmaceutically acceptable salt thereof, or composition, and (b) (e.g. an effective amount of) at least one second active agent selected from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, SRX3207, R09021, gusacitinib, R112, PRT-060318, and OXSI-2, optionally wherein such at least one second active agent is formulated as a pharmaceutical composition comprising a pharmaceutically acceptable carrier or excipient. The compound, pharmaceutically acceptable salt thereof, or pharmaceutical composition and the at least one second active agent can be administered by the same route. The compound, pharmaceutically acceptable salt thereof, or pharmaceutical composition and the at least one second active agent can be administered by different routes. In certain embodiments, the at least one second active agent comprises hydroxyurea. In certain embodiments, the at least one second active agent comprises more than one second active agent which are all selected from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, SRX3207, R09021, gusacitinib, R112, PRT-060318, and OXSI-2, optionally wherein the at least one active agent is formulated as a pharmaceutical composition comprising a pharmaceutically acceptable carrier or excipient.

Still further provided is a method of treating a subject for a disease involving the release of microvesicles from blood cells. The method comprises administering an effective amount of a compound or a pharmaceutically acceptable salt thereof, in which the compound comprises a rigid, fused bicyclic ring, which is substituted with at least two substituents, at least one of which is an N-linked, optionally substituted, carbo- or heterocycle and the other of which is an indole substituted with at least one substituent comprising a 4-6-membered, optionally substituted, carbo- or heterocycle. The disease can be thalassemia. The thalassemia can be α-thalassemia or β-thalassemia, alone or in combination with sickle cell disease. The disease can be glucose-6-phosphate dehydrogenase deficiency. The disease can be glutathione reductase deficiency.

The method can further comprise the simultaneous or sequential administration, in either order, of compound or a pharmaceutically acceptable salt thereof and (e.g., an effective amount of) a Src inhibitor, and optionally wherein the Src inhibitor is formulated as a pharmaceutical composition comprising a pharmaceutically acceptable carrier or excipient. The Src inhibitor and compound (or pharmaceutically acceptable salt thereof) can be administered by the sane or different routes. The Src inhibitor can be dasatinib, ibrutinib, bafetinib, PP1, PP2, PP121, or a combination of two or more of the foregoing.

Even still further provided is a method of treating sickle cell disease in a subject, which method comprises administering an effective amount of a compound or a pharmaceutically acceptable salt thereof:
(i) which comprises a pyrazine, which is substituted with at least three substituents, at least one of which is a carboxamide or a lactam, another of which is an N-linked, optionally substituted, carbo- or heterocycle, and another of which is a carbo- or heterocycle with at least one substituent, at least one of which is a 4-6-membered, optionally substituted, carbo- or heterocycle,
(ii) which inhibits Syk tyrosine kinase from phosphorylating the human erythrocyte anion transporter band 3,
(iii) which inhibits Syk tyrosine kinase at a concentration that is at least about 20-fold lower than the concentration at which the compound inhibits HGFR, EGFR, FGFR3, IGF1R, and/or a VEGFR,
wherein, additionally or alternatively to (ii) and (iii), the compound of (i) has:
(iv) a CLogP value between about 2 and about 5 (e.g., between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds,
where upon the subject is treated for sickle cell disease. The compound (or pharmaceutically acceptable salt thereof) can inhibit Syk tyrosine kinase from phosphorylating tyrosine 8 and tyrosine 21 in the human erythrocyte anion transporter band 3. The compound (or pharmaceutically acceptable salt thereof) can stabilize erythrocyte cell membranes and reduce the release of microvesicles from sickle cells. The compound (or pharmaceutically acceptable salt thereof) can stabilize erythrocyte cell membranes and reduce the release of hemoglobin from sickle cells. The risk of vaso-occlusive crisis in the subject can be reduced. The compound (or pharmaceutically acceptable salt thereof) can inhibit Syk tyrosine kinase at an IC₅₀ of 200 nM or lower. The subject can be a human child.

The method can further comprise the simultaneous or sequential administration, in either order, of (a) the compound (or pharmaceutically acceptable salt thereof) and (b) one or more of fostamatinib, PRT062607, TAK-659, TAE-684, entospletinib, lanraplenib, cerdulatinib, piceatannol, S701. Syk II, Syk IV, TAS05567, GSK143, Syk-IN-3, Syk-IN-4, SRX3207, R09021, gusacitinib, R112, PRT-060318, and OXSI-2, optionally as a pharmaceutical composition comprising a pharmaceutically acceptable carrier or excipient. The one or more other compounds can be administered by the same or different route.

A method of treating a subject for a disease involving the release of microvesicles from blood cells is also provided. The method comprises administering an effective amount of a compound (or pharmaceutically acceptable salt thereof), which comprises a pyrazine, which is substituted with at least three substituents, at least one of which is a carboxamide or a lactam, another of which is an N-linked, optionally substituted, carbo- or heterocycle, and another of which is a carbo- or heterocycle with at least one substituent, at least one of which is a 4-6-membered, optionally substituted, carbo- or heterocycle, whereupon the subject is treated for the disease involving the release of microvesicles. The disease can be thalassemia. The thalassemia can be α-thalassemia or β-thalassemia, alone or in combination with sickle cell disease. The disease can be glucose-6-phosphate dehydrogenase deficiency. The disease can be glutathione reductase deficiency. The method can further comprise the simultaneous or sequential administration, in either order of the compound (or pharmaceutically acceptable salt thereof) and a Src inhibitor, optionally as a pharmaceutical composition comprising a pharmaceutically acceptable carrier or excipient. The Src inhibitor can be administered by the sane or different route as the compound (or pharmaceutically acceptable salt thereof). The Src inhibitor can be dasatinib, ibrutinib, bafetinib, PP1, PP2, PP121, or a combination of two or more of the foregoing.

### SEQUENCE LISTING

The sequence herein (SEQ ID NO: 1) is also provided in computer readable form encoded in a file filed herewith and incorporated herein by reference. The information recorded in computer readable form is identical to the written Sequence Listing provided below, pursuant to 37 C.F.R.

SEQ ID NO: 1 is the amino acid sequence of a
spleen tyrosine kinase (Syk) protein:
MASSGMADSANHLPFFFGNITREEAEDYLVQGGMSDGLYLLRQSRNYLG
GFALSVAHGRKAHHYTIERELNGTYAIAGGRTHASPADLCHYHSQESDG
LVCLLKKPFNRPQGVQPKTGPFEDLKENLIREYVKQTWNLQGQALEQAI
ISQKPQLEKLIATTAHEKMPWFHGKISREESEQIVLIGSKTNGKFLIRA
RDNNGSYALCLLHEGKVLHYRIDKDKTGKLSIPEGKKFDTLWQLVEHYS
YKADGLLRVLTVPCQKIGTQGNVNFGGRPQLPGSHPATWSAGGIISRIK
SYSFPKPGHRKSSPAQGNRQESTVSFNPYEPELAPWAADKGPQREALPM
DTEVYESPYADPEEIRPKEVYLDRKLLTLEDKELGSGNFGTVKKGYYQM
KKVVKTVAVKILKNEANDPALKDELLAEANVMQQLDNPYIVRMIGICEA
ESWMLVMEMAELGPLNKYLQQNRHVKDKNIIELVHQVSMGMKYLEESNF
VHRDLAARNVLLVTQHYAKISDFGLSKALRADENYYKAQTHGKWPVKWY
APECINYYKFSSKSDVWSFGVLMWEAFSYGQKPYRGMKGSEVTAMLEKG
ERMGCPAGCPREMYDLMNLCWTYDVENRPGFAAVELRLRNYYYDVVN.

The content of the XML file of the sequence listing (named “PRF69753-03SeqListingXML.xml” which is 2.7 kb in size, created Mar. 13, 2023, and electronically submitted via Patent Center on Mar. 14, 2023) is incorporated herein by reference in its entirety. The information recorded in computer readable form is identical to the written Sequence Listing provided herein (on paper) pursuant to 37 C.F.R. § 1.821(f).

### DETAILED DESCRIPTION

While the concepts of the present disclosure are illustrated and described in detail in the description herein, results in the description are to be considered as exemplary and not restrictive in character; it being understood that only the illustrative embodiments are shown and described, and that all changes and modifications that come within the spirit of the disclosure are desired to be protected.

The present disclosure is predicated, at least in part, on the unprecedented discovery of a compound comprising a small molecule inhibitor that inhibits spleen tyrosine kinase (Syk) without substantially disrupting other pathways that provide regulated growth factor kinases related to the growth development of a subject. The compound can be used to treat diseases of the red blood cells (RBCs) such as hemoglobinopathies (e.g., sickle cell disease (SCD) and thalassemia) and other RBC-mediated diseases such as glucose-6-phosphate dehydrogenase (G6PD) deficiency and glutathione reductase deficiency.

Phosphorylation/dephosphorylation of protein tyrosine residues has been implicated in the regulation of several erythrocyte functions including cell shape. The aforementioned diseases of the RBCs involve a mechanism in which elevated tyrosine phosphorylation of Band 3 initiates sequelae that cause vaso-occlusion and the disease symptoms. In this mechanism, accelerated denaturation of sickle hemoglobin (HbS), hemichrome formation, and release of heme can collectively induce oxidative stress within the RBC. Increased oxidative stress can then cause inhibition of erythrocyte/RBC tyrosine phosphatases, which normally prevent constitutive Band 3 tyrosine phosphorylation. Upon inhibition of these phosphatases, over-phosphorylation of Band 3 then induces global destabilization of the erythrocyte membrane, accelerating intravascular hemolysis and microparticle (MP) release. The increased plasma hemoglobin and heme can also “activate” the vascular endothelium, which causes expression of adhesion receptors (e.g., p-selectin, E-selectin, and von Willebrand factor), as well as sequestration of the vasodilator (NO), while the release of MPs can trigger intravascular thrombosis via activation of prothrombin.

Ultimately, these processes can promote vaso-occlusive events and result in the symptoms of SCD and other RBC diseases, such as, for example, tissue hypoxia, ischemia-reperfusion injury, organ damage and associated morbidities, and debilitating pain which results in significant suffering and can require medical treatment and/or hospitalization. When initiated concomitantly with the loss of RBC deformability and enhanced vaso-adhesion, the sequelae associated with membrane weakening can aggravate an already compromised blood flow, leading to micro-emboli and progressive tissue damage. Sickle cell hemolysis, reduced sickle red cell lifespan, and anemia can further aggravate clinical symptoms.

At least one of the kinases involved in the phosphorylation of Band 3 is Syk (p72^{syk}). Syk is a nonreceptor protein tyrosine kinase that is expressed in substantial amounts in RBCs. For purposes of discussion of amino acids in Syk, reference is made to the long isoform (Uniprot ID P43405-1), which has SEQ ID NO: 1. Alternative splicing results in the deletion of amino acids 283-305 to give the short isoform (Uniprot ID P43405-2).

Syk binds to the cytoplasmic region of cells bearing Feγ-activating receptors that contain the immunoreceptor tyrosine-based activation motif (ITAM). Receptor binding results in phosphorylation, which leads to Syk activation by increasing its autophosphorylating activity. Syk activations activates various downstream kinases which ultimately result in proinflammatory cytokine release and the cascade mentioned above.

### Compounds

The compounds hereof comprise a small molecule inhibitor that inhibits Syk (i.e., bind to and suppress the Syk receptor), and can thereby inhibit and/or prevent this cascade by suppressing Band 3 tyrosine phosphorylation in the affected RBCs. In addition, the compounds can suppress MP and hemoglobin release from the affected RBCs.

In certain embodiments, the compound is of formula X:

A-B-C (X)

or a pharmaceutically acceptable salt thereof, wherein:
A is a first ring that is nitrogen (N)-linked and optionally substituted, the first ring comprising a fused bicyclic ring or a monocyclic ring with each ring of the first ring comprising a 4-6-membered heterocycle or carbocycle:
B is a second ring that is optionally substituted with at least one substituent, the second ring comprising a fused bicyclic or tricyclic ring or a monocyclic ring, and wherein each ring of the second ring comprises a 5- or 6-membered heterocycle or carbocycle; and
C is a third bicyclic ring comprising a 5- or 6-membered carbocycle fused with a 5- or 6-membered carbocycle or heterocycle, wherein the third bicyclic ring is substituted with at least:

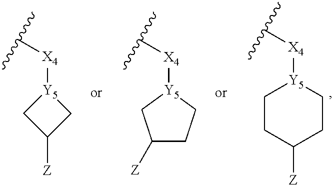

or an amine, or a ketone, or a carboxamide, or an alcohol (e.g., a propanol),
wherein:
   X₄ is CH₂, NH, CO, O, or S,
   Y₅ is CH or N,
   Z is selected from OH, SH, NH₂, CO₂H, NHCH₃, and

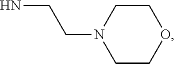

and [symbol] is a point of attachment.

A of the compound can be:

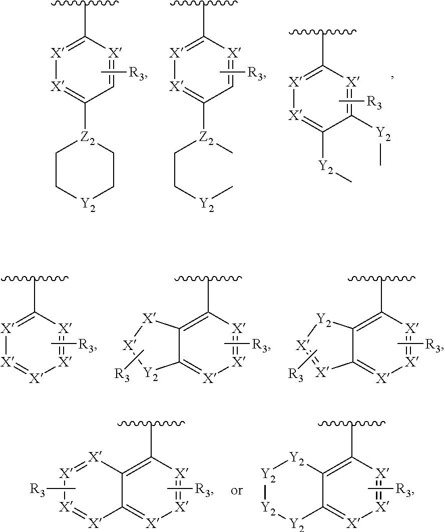

or a pharmaceutically acceptable salt of any of the foregoing structures, wherein:
each X′ is independently selected from CH and NH;
each Y₂ is independently selected from CH₂, NH, O, and S;
Z₂ is CH or N; and
each R₃ is independently selected from H, F, Cl, O, and —Y—(CH₂)ₙ—, in which n=1-14.

A of the compound can be

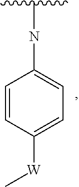

wherein [symbol] is a point of attachment to B, and W is O, NH, or SH.

A of the compound can be

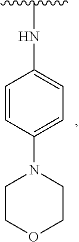

wherein [symbol] is a point of attachment to B.

A of the compound can be

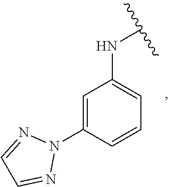

wherein [symbol] is a point of attachment to B.

The N-linked to the first ring of A (e.g., a fused bicyclic ring or a monocyclic ring) can form a nitrogen bridge with B of the compound.

The first ring of A can comprise a 4-6-membered heterocycle (e.g., containing one or more of N, O, or S). The first ring of A can comprise a 4-6-membered carbocycle. The first ring of A can be a fused bicyclic ring comprising two 6-membered rings. The first ring of A can be a fused bicyclic ring comprising a 6-membered ring and a 5-membered ring.

In certain embodiments, A is substituted (e.g., the first ring of A is substituted). A can be a monocyclic ring substituted with a bulky substituent para to the nitrogen bridge. The compound can have an increased docking score for Syk tyrosine kinase as compared to an otherwise identical compound lacking the bulky substituent para to the nitrogen bridge (see Table 1 below).

In certain embodiments, the bulky substituent is morpholine, for example, having the structure:

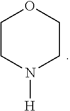

The first ring of A can be or comprise a monocyclic carbocycle (e.g., that is 6-membered). In certain embodiments, the first ring of A comprises benzene.

In certain embodiments, the first ring of A is benzene that is substituted with at least two substituents. The first ring of A can be or comprise a monocyclic carbocycle (e.g., that is 6-membered) that is optionally substituted (e.g., substituted with two methoxy groups). The first ring of A can comprise a closed ring. The first ring of A can comprise a first closed ring (e.g., a carbocycle) fused with a second open ring. The first ring of A can be bicyclic and comprise a first closed ring (e.g., a 6-membered carbocycle) fused with a second closed ring (e.g., a 5-membered carbocycle or heterocycle), either or neither of which can be substituted.

As noted above, the first ring(s) of A can be optionally substituted with one or more substituents, each independently selected from the group consisting of hydrogen, deuterium, halo, azido, cyano, nitro, hydroxy, amino, thio, carboxy, ester, amide and derivatives thereof alkyl, alkenyl, alkynyl, heteroalkyl, heteroalkenyl, heteroalknyl, heterocyclyl, cycloalkyl, cycloalkenyl, cycloheteroalkyl, cycloheteroalkenyl, acyl, aryl, heteroaryl, arylalkyl, arylalkenyl, arylalkynyl, morpholine, and nitrogen heterocycle.

In certain embodiments, the compound has the structure of:

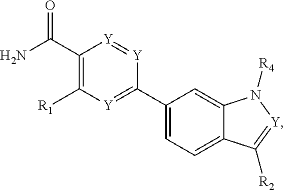

or is a pharmaceutically acceptable salt thereof, wherein

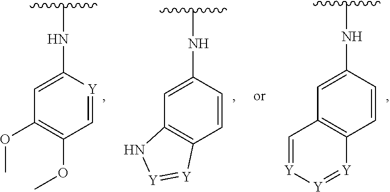

R₁ is
R₂ is

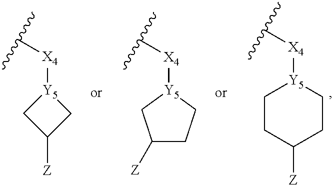

or an amine, or an alcohol (e.g., a propanol),
R₄ is H, CH₃ an amine, or a methanol
X₄ is independently CH₂, NH, CO, O, or S,
each Y and Y₅ is each independently CH or N,
Z is selected from OH, SH, NH₂, CO₂H, NHCH₃, and

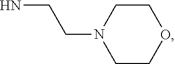

and
[symbol] is a point of attachment.

B of the compound can be a second ring, optionally substituted with at least one substituent, wherein the second ring is monocyclic or comprises a fused bicyclic or fused tricyclic ring. Each ring of the second ring can comprise a 5- or 6-membered carbocycle or heterocycle.

The one or more substituents of B can each be independently selected from the group consisting of hydrogen, deuterium, amide and derivatives thereof, carbonyl, and an alkyl.

B of the compound can be rigid.

B can be a monocyclic ring, optionally substituted with an amide. B can be pyridine. B can be pyrazine. B can be pyrazine substituted with at least one substituent. B can comprise imidazole substituted with at least an amide.

B can be a fused bicyclic ring, optionally substituted (e.g., optionally substituted with an amide or a carbonyl group). In certain embodiments, the fused bicyclic ring comprises a pyrimidine fused with a N-containing 5-membered heterocycle that is optionally substituted.

In certain embodiments, B is:

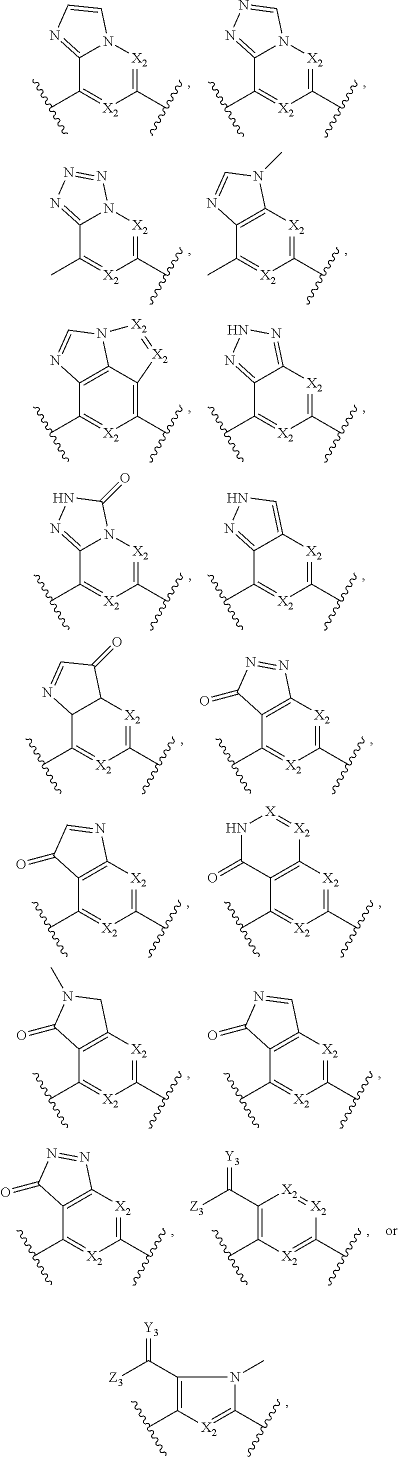

wherein
each X₂ is independently selected from CH and N,
Y₃ is selected from O, NH and S; and
Z₃ is selected from OH, NH₂, and NHCH₃.

In certain embodiments, B of the compound is not

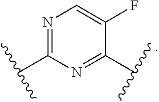 [SMILES: *c1ncc(F)c(*)n1]

For example, B of the compound can be or comprise:

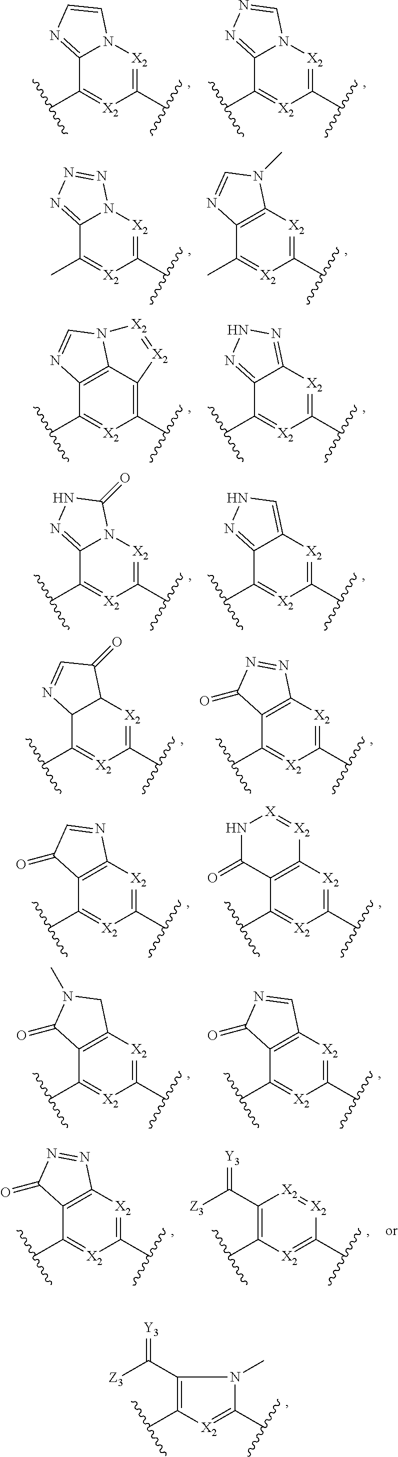

wherein:
each X₂ is independently selected from CH and N;
Y₃ is selected from O, NH and S; and
Z₃ is selected from OH, NH₂, and NHCH₃, with the proviso that B is not:

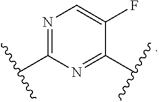 [SMILES: *c1ncc(F)c(*)n1]

When the compound (or pharmaceutically acceptable salt thereof) is administered to a subject, B can form a hydrogen bond with the amino acid residue Ala451 in the catalytic domain of Syk tyrosine kinase. Additionally, Z of the first substituent of B can form a hydrogen bond with amino acid residue Asp512 in the catalytic domain of Syk tyrosine kinase.

C of the compound comprises a third bicyclic ring. The third ring can comprise a 5- or 6-membered carbocycle fused to a 5- or 6-membered carbocycle or heterocycle. In certain embodiments, C comprises a third bicyclic ring having a 6-membered carbocycle fused to a 5-membered heterocycle (e.g., a N-containing heterocycle) or a carbocycle. In certain embodiments, C comprises a third bicyclic ring having a 6-membered carbocycle fused to a 6-membered heterocycle (e.g., a N-containing heterocycle) or a carbocycle. The bicyclic ring of C can be an indole.

The third fused bicyclic ring is substituted with at least one substituent (e.g., the first substituent). C can comprise the following structure:

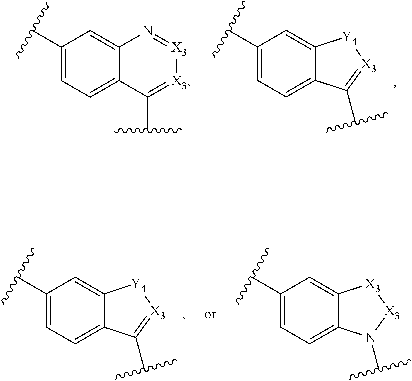

wherein each X₃ is independently selected from CH and N; Y₄ is CH₂, NH, O, or SH and [symbol] is the point of attachment of the at least one substituent of the third ring (C).

In certain embodiments, the third bicyclic ring of C is substituted with at least a first substituent comprising:

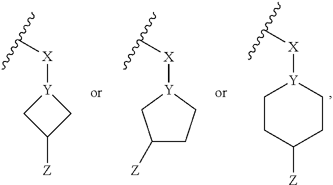

or an amine, or a ketone, or a carboxamide, or an alcohol (e.g., a propanol), wherein:
X is each independently CH₂, NH, CO, O, or S,
Y is each independently CH or N,
Z is selected from OH, SH, NH₂, CO₂H, NHCH₃, and

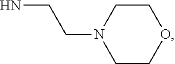

and
[symbol] is a point of attachment (e.g., to the 5- or 6-membered carbocycle or heterocycle of C of the compound).

In certain embodiments, Z of the first substituent is —OR in which R is

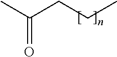

and n=1-14.

In certain embodiments, C comprises the following structure:

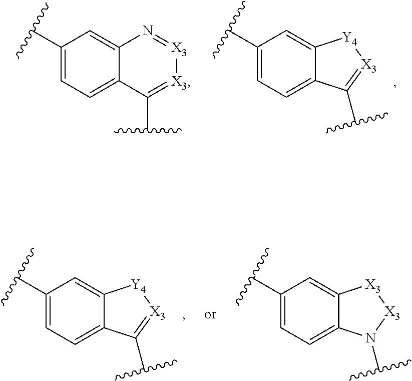

wherein each X₃ is independently selected from CH and N; Y is CH₂, NH, O or SH; and [symbol] is the point of attachment to a first substituent, wherein the first substituent of the third bicyclic ring of C is:

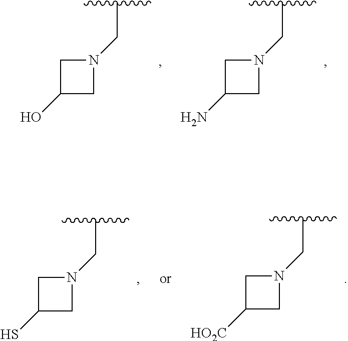 [SMILES: *CN1CC(O)C1 ; *CN1CC(N)C1 ; *CN1CC(S)C1 ; *CN1CC(C(=O)O)C1]

The bicyclic ring of C can be an indole and the first substituent of C can comprise a 4-6-membered, optionally substituted, carbo- or heterocycle substituent. In certain embodiments, the first substituent of C is a lipophilic ester. In certain embodiments, the bicyclic ring of C is an indole and the first substituent of C is a lipophilic ester.

Z of the first substituent of C can comprise a phosphate or phosphonate ester. In certain embodiments, the substituent of C is

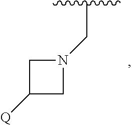

wherein: Q is a phosphate or phosphonate ester; or Q is RC(═O)O—, in which R is an alkyl.

C can also comprise at least a second substituent. In certain embodiments, C is substituted with CH₃, H, CH₂OH, an amine, and/or an alkyl.

One or more of the hydrogens (H) of the compound can be optionally substituted with deuterium.

In certain embodiments, (e.g., when administered to a subject) B of the compound can form a hydrogen bond with amino acid residue Ala451 in a catalytic domain of Syk tyrosine kinase and the first substituent of C can form a hydrogen bond with amino acid residue Asp512 in a catalytic domain of Syk tyrosine kinase.

The compound can have a CLogP value between about 2 and about 5 (e.g., between 2 and about 5, about 2 and 5, or 2 and 5). The compound can have a polar surface area of less than about 250 square Å (e.g., less than 250 square Å). The compound can have no more than 5 H-bond donors. The compound can have no more than 15 H-bond acceptors. The compound can have no more than 20 rotatable bonds. In certain embodiments, the compound can have a CLogP value between about 2 and about 5 (e.g., between 2 and about 5, about 2 and 5, or 2 and 5); a polar surface area of less than about 250 square Å (e.g., less than 250 square Å) no more than 5 H-bond donors; no more than 15 H-bond acceptors; and/or no more than 20 rotatable bonds.

The compound can bind to Syk tyrosine kinase with specificity. When administered to a subject, in certain embodiments, the compound inhibits Syk from phosphorylating an erythrocyte (e.g., a human erythrocyte) anion transporter Band 3.

The compound can comprise the structure of Formula (XX):

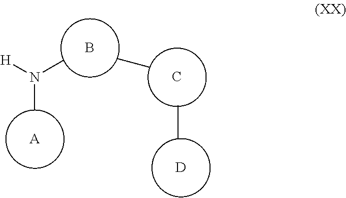

or be a pharmaceutically acceptable salt thereof, in which ring A is

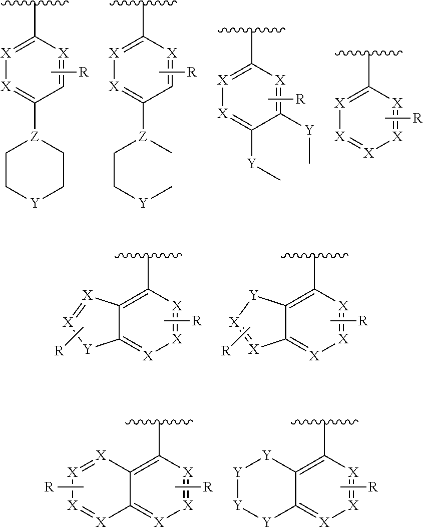

wherein:
   each X is independently selected from CH and NH,
   each Y is independently selected from CH₂, NH, O, and S,
   Z is CH or N, and
   each R is independently selected from H, F, Cl, OH, and —Y—(CH₂)ₙ— in which n=1-14;
      in which ring B is

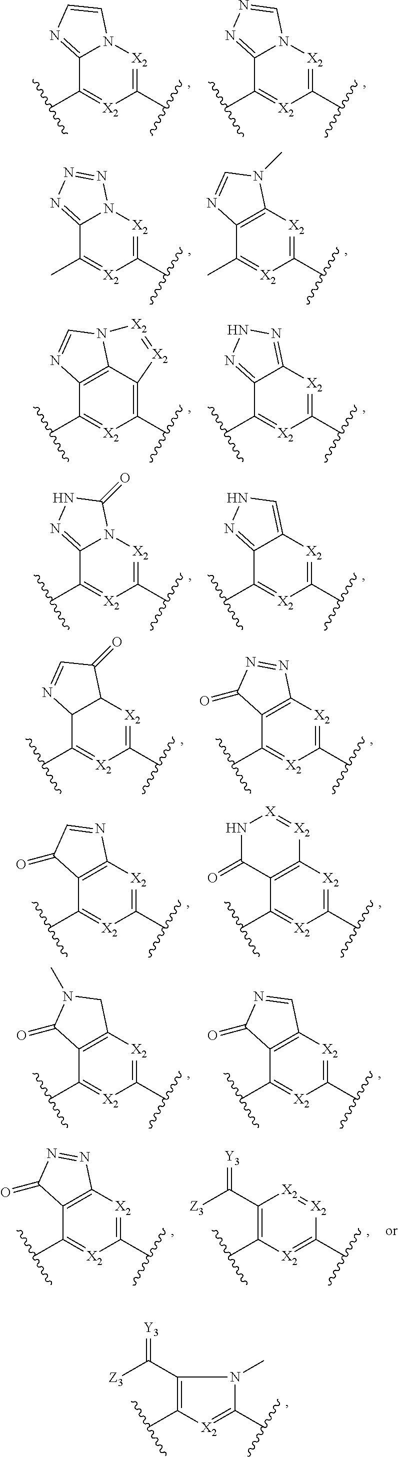

wherein
   each X₂ is independently selected from CH and N;
   Y₃ is selected from O, NH and S; and
   Z₃ is selected from OH, NH₂, and NHCH₃, with the proviso that B is not

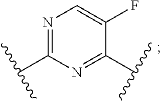 [SMILES: *c1ncc(F)c(*)n1]

in which ring C is

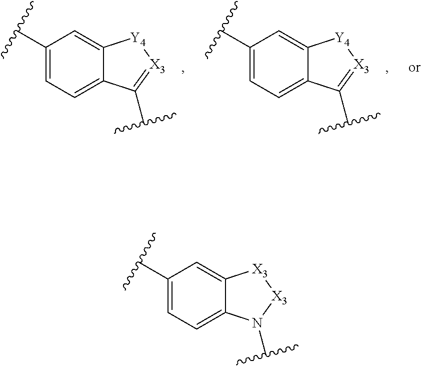

wherein:
   each X₃ is independently selected from CH and N, and
   Y₄ is selected from CH₂, NH, O, and SH and
      in which ring D is

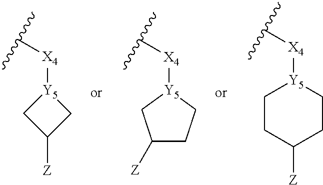

wherein:
   X₄ is selected from CH₂, NH, CO, O, and S,
   Y₅ is selected from CH and N;
   Z is selected from OH, SH, NH₂, NHCH₃, and

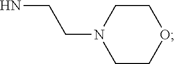

and
wherein one or more hydrogens can be optionally substituted with deuterium.

Rings A, B, C, and D of Formula (XX) can comprise any of the aforementioned embodiments of A, B, and C of the compounds of Formula (X) described above. For example, ring A of Formula (XX) can be substituted with a bulky substituent para to the nitrogen bridge. The bulky substituent can be a 6-membered, optionally substituted, open or closed, carbo- or hetero-cycle. The bulky substituent can be morpholine. Additionally, ring D of Formula (XX) can comprise any of the embodiments of the first substituent of C of Formula (X).

The compound can comprise a structure of:

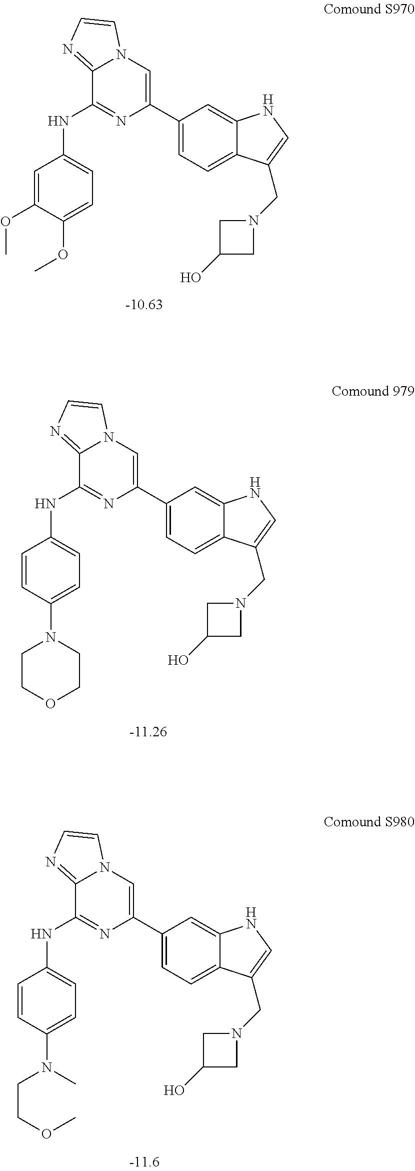 [SMILES: COc1ccc(Nc2nc(-c3ccc4c(CN5CC(O)C5)c[nH]c4c3)cn3ccnc23)cc1OC ; OC1CN(Cc2c[nH]c3cc(-c4cn5ccnc5c(Nc5ccc(N6CCOCC6)cc5)n4)ccc23)C1 ; COCCN(C)c1ccc(Nc2nc(-c3ccc4c(CN5CC(O)C5)c[nH]c4c3)cn3ccnc23)cc1]

(with the docking score for Syk tyrosine kinase shown directly below each of the compounds)
or is a pharmaceutically acceptable salt of any of the foregoing.

The compound can comprise the structure:

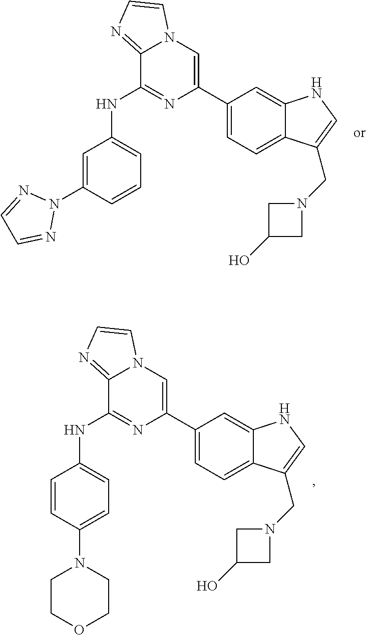 [SMILES: OC1CN(Cc2c[nH]c3cc(-c4cn5ccnc5c(Nc5cccc(-n6nccn6)c5)n4)ccc23)C1 ; OC1CN(Cc2c[nH]c3cc(-c4cn5ccnc5c(Nc5ccc(N6CCOCC6)cc5)n4)ccc23)C1]

or be a pharmaceutically acceptable salt of any of the foregoing.

The compound can comprise the structure:

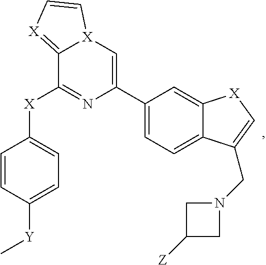

or be a pharmaceutically acceptable salt thereof, in which each X is independently selected from CH₂, N, NH, O, and S; Y is O, NH, or SH; and Z is OH or NH₂. The 4-6-membered, optionally substituted, carbo- or heterocycle substituent on the indole can comprise a lipophilic ester. The Z on ring D can be —OR in which R is

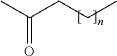

and n=1-14.

The compound can be a compound (or pharmaceutically acceptable salt thereof) which comprises a pyrazine, which is substituted with at least three substituents, at least one of which is a carboxamide or a lactam, another of which is an N-linked, optionally substituted, carbo- or heterocycle, and another of which is a carbo- or heterocycle with at least one substituent, at least one of which is a 4-6-membered, optionally substituted, carbo- or heterocycle.

The compound can have the structure of:

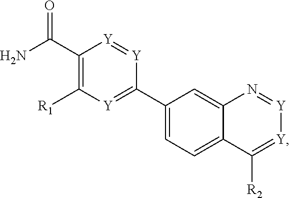

or be a pharmaceutically acceptable salt thereof wherein:
R₁ is

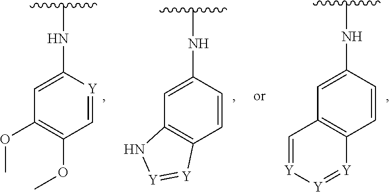

R₂ is

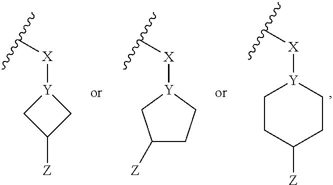

or an amine, or an alcohol (e.g., a propanol),
X is each independently CH₂, NH CO, O, or S,
Y is each independently CH or N,
Z is selected from OH, SH, NH₂, CO₂H, NHCH₃, and

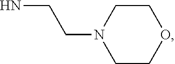

and
[symbol] is a point of attachment.

In certain embodiments, the compound comprises:

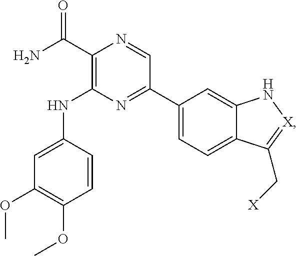

or a pharmaceutically acceptable salt thereof, wherein X is CH or N, and R is

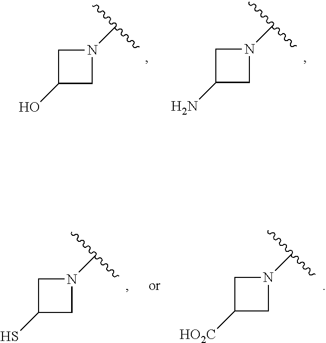 [SMILES: *N1CC(O)C1 ; *N1CC(N)C1 ; *N1CC(S)C1 ; *N1CC(C(=O)O)C1]

In certain embodiments, the compound comprises:

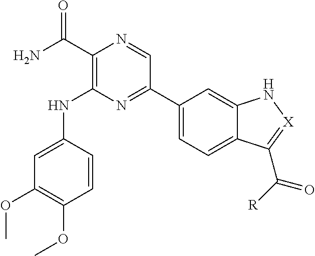

or is a pharmaceutically acceptable salt thereof, wherein X is CH or N, and R is

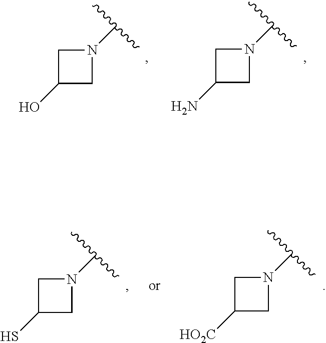 [SMILES: *N1CC(O)C1 ; *N1CC(N)C1 ; *N1CC(S)C1 ; *N1CC(C(=O)O)C1]

In certain embodiments, the compound comprises:

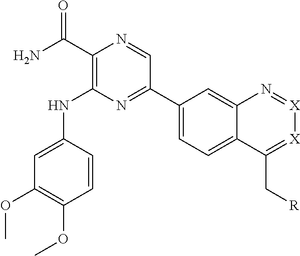

or is a pharmaceutically acceptable salt thereof, wherein X is CH or N, and R is

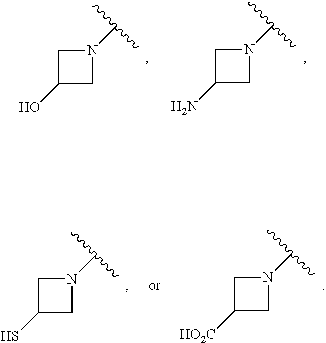 [SMILES: *N1CC(O)C1 ; *N1CC(N)C1 ; *N1CC(S)C1 ; *N1CC(C(=O)O)C1]

In certain embodiments, the compound comprises:

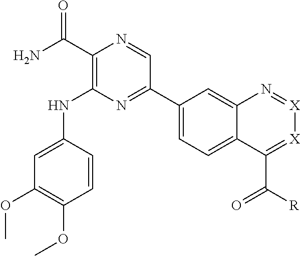

or is a pharmaceutically acceptable salt thereof wherein X is CH or N and R is

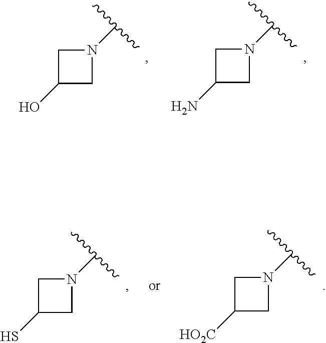 [SMILES: *N1CC(O)C1 ; *N1CC(N)C1 ; *N1CC(S)C1 ; *N1CC(C(=O)O)C1]

In certain embodiments, the compound comprises:

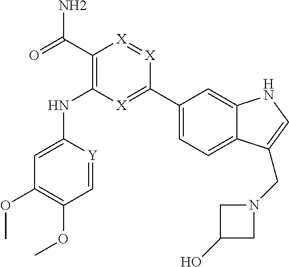

or is a pharmaceutically acceptable salt thereof, wherein each X is independently CH or N, and Y is CH or N. In certain embodiments, when each X and Y are all CH, the compound can exhibit a weaker binding affinity to Syk tyrosine kinase as compared to when at least one X and/or Y are not CH.

In certain embodiments, the compound comprises:

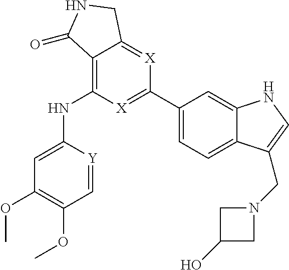

or is a pharmaceutically acceptable salt thereof, wherein each X is independently CH or N, and Y is CH or N. In certain embodiments, when each X and Y are all CH, the compound can exhibit a weaker binding affinity to Syk tyrosine kinase as compared to when at least one X and/or Y are not CH.

The compound can comprise the structure:

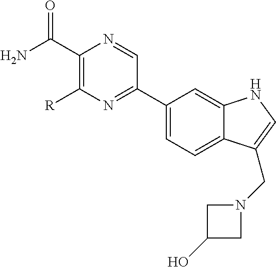

or be a pharmaceutically acceptable salt thereof wherein:
R

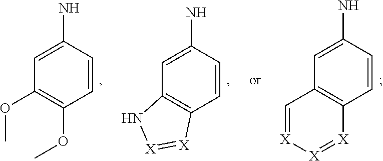

and
X is independently CH or N.

The compound can comprise the structure:

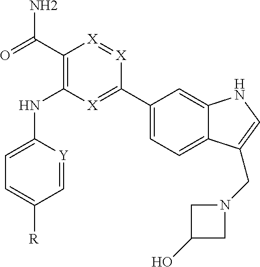

or be a pharmaceutically acceptable salt thereof, wherein:
R is carbo- or heterocyclic;
X is independently CH or N; and
Y is CH or N.

The compound can comprise the structure:

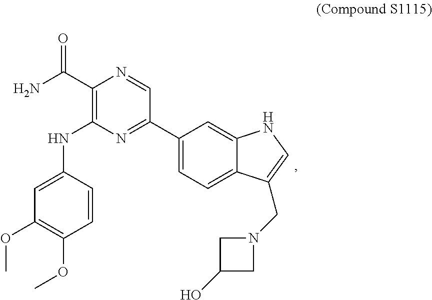 [SMILES: COc1ccc(Nc2nc(-c3ccc4c(CN5CC(O)C5)c[nH]c4c3)cnc2C(N)=O)cc1OC]

or be a pharmaceutically acceptable salt thereof.

In certain embodiments the hydroxyl (HO—) group of the first substituent of C of Formula (X) or of ring D of Formula (XX) can be replaced with phosphate or a phosphonate ester. For example, the first substituent of the bicylic ring of C can b

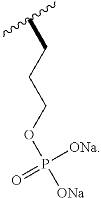

For example, see Compound S2053P. Such a change can improve compound solubility and/or cell permeability. In alternative embodiments, the hydroxyl (HO—) group can be replaced with RC(═O)O— in which R is an alkyl (e.g., a C₁-C₆ alkyl). Such a change can improve cell permeability.

The compound can have the structure:

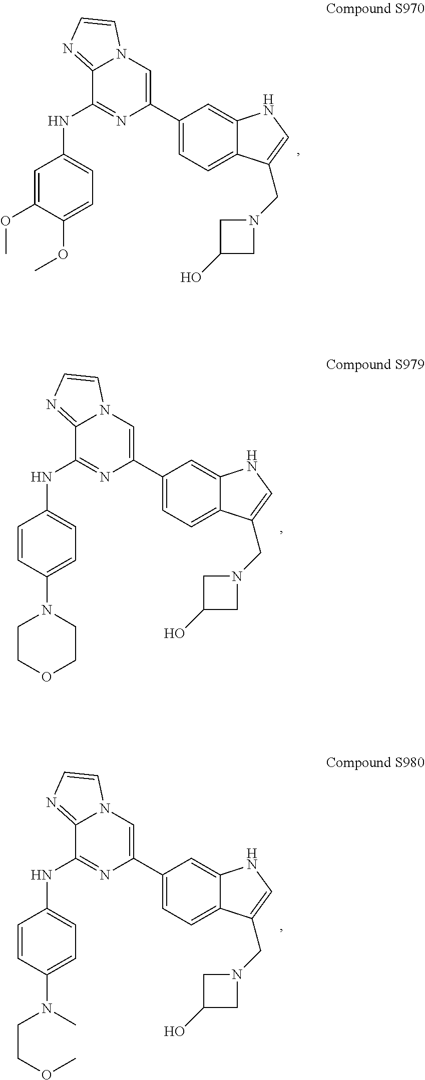 [SMILES: COc1ccc(Nc2nc(-c3ccc4c(CN5CC(O)C5)c[nH]c4c3)cn3ccnc23)cc1OC ; OC1CN(Cc2c[nH]c3cc(-c4cn5ccnc5c(Nc5ccc(N6CCOCC6)cc5)n4)ccc23)C1 ; COCCN(C)c1ccc(Nc2nc(-c3ccc4c(CN5CC(O)C5)c[nH]c4c3)cn3ccnc23)cc1]

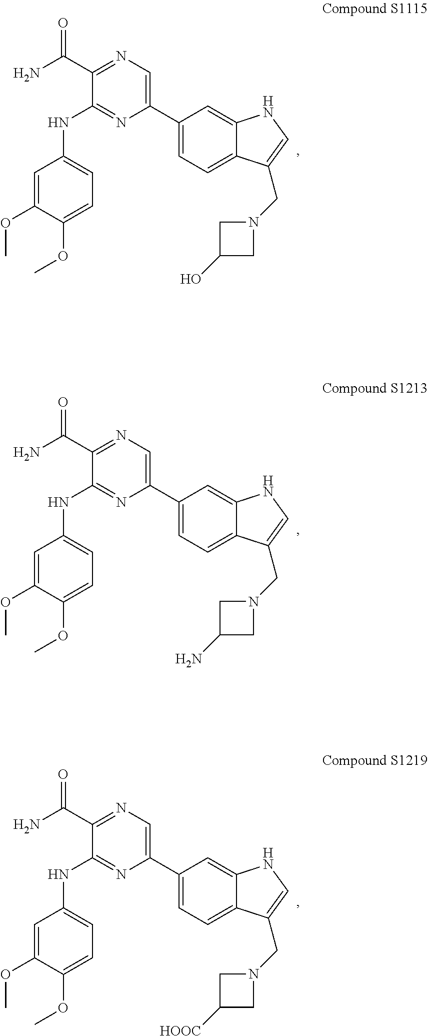 [SMILES: COc1ccc(Nc2nc(-c3ccc4c(CN5CC(O)C5)c[nH]c4c3)cnc2C(N)=O)cc1OC ; COc1ccc(Nc2nc(-c3ccc4c(CN5CC(N)C5)c[nH]c4c3)cnc2C(N)=O)cc1OC ; COc1ccc(Nc2nc(-c3ccc4c(CN5CC(C(=O)O)C5)c[nH]c4c3)cnc2C(N)=O)cc1OC]

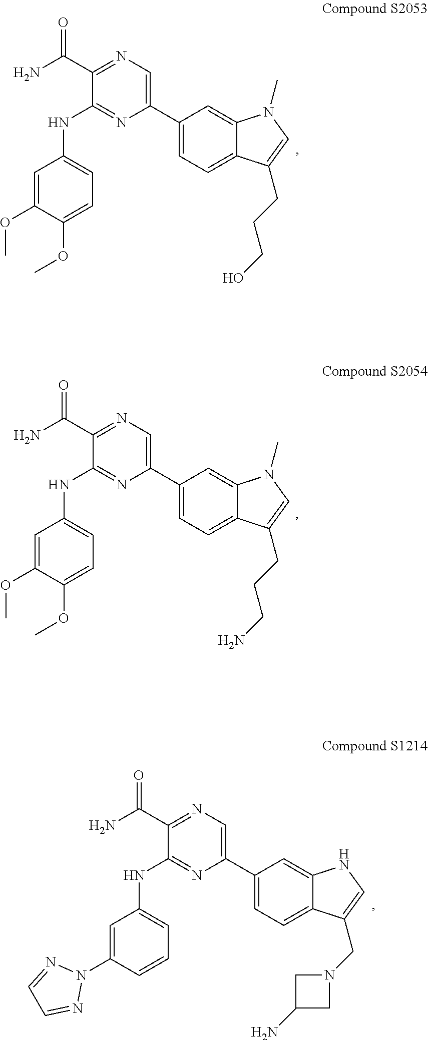 [SMILES: COc1ccc(Nc2nc(-c3ccc4c(CCCO)cn(C)c4c3)cnc2C(N)=O)cc1OC ; COc1ccc(Nc2nc(-c3ccc4c(CCCN)cn(C)c4c3)cnc2C(N)=O)cc1OC ; NC(=O)c1ncc(-c2ccc3c(CN4CC(N)C4)c[nH]c3c2)nc1Nc1cccc(-n2nccn2)c1]

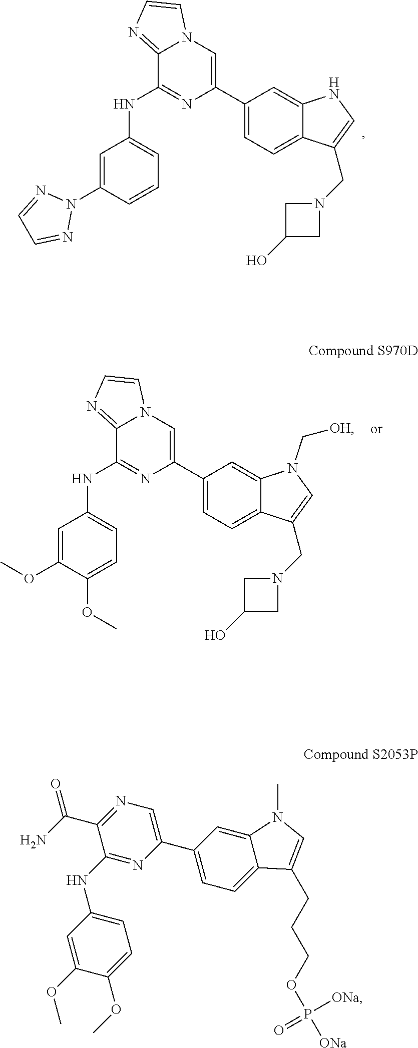

or be a pharmaceutically acceptable salt of any of the foregoing structures.

The compound can further comprise a phosphate prodrug version. In certain embodiments, for example, the compound is:

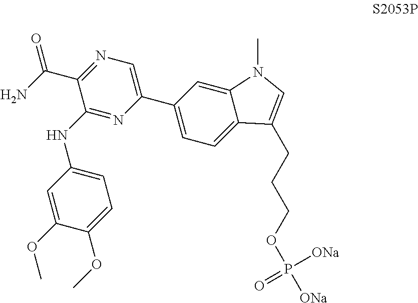

or is a pharmaceutically acceptable salt thereof.

The compound can have or comprise the structure

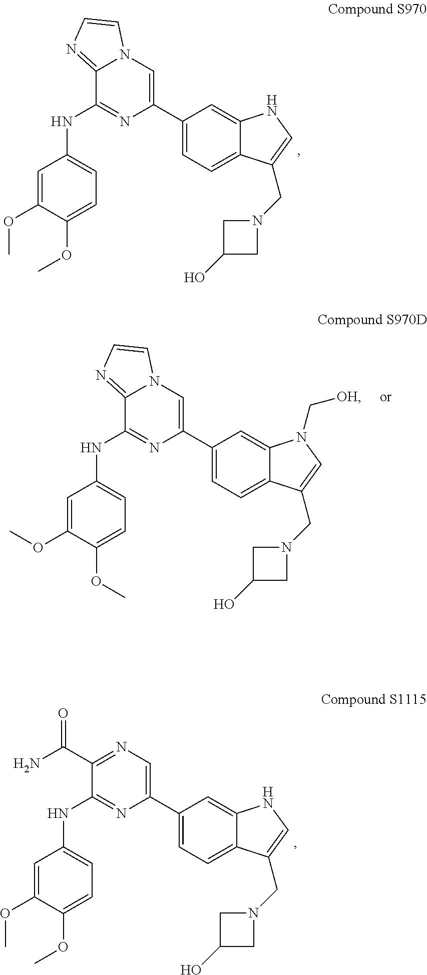 [SMILES: COc1ccc(Nc2nc(-c3ccc4c(CN5CC(O)C5)c[nH]c4c3)cn3ccnc23)cc1OC ; COc1ccc(Nc2nc(-c3ccc4c(CN5CC(O)C5)cn(CO)c4c3)cn3ccnc23)cc1OC ; COc1ccc(Nc2nc(-c3ccc4c(CN5CC(O)C5)c[nH]c4c3)cnc2C(N)=O)cc1OC]

or be a pharmaceutically acceptable salt of any of the foregoing structures.

In certain embodiments, the compound (or pharmaceutically acceptable salt thereof) comprises a rigid, fused bicyclic ring, which is substituted with at least two substituents, at least one of which is an N-linked, optionally substituted, carbo- or heterocycle and the other of which is an indole substituted with at least one substituent, at least one of which is a 4-6-membered, optionally substituted, carbo- or heterocycle.

As noted above, the compounds can be Syk-specific inhibitors. The compound can bind to a Syk receptor with specificity, thus reducing off-target interactions, for example, with growth factor receptors. “Binds with specificity,” “binds with high affinity,” or “specifically” or “selectively” binds, when referring to a small molecule/receptor or other binding pairs indicates that, under designated conditions, a specified compound or recognition region thereof binds to a particular receptor or site (e.g., a Syk inhibition site such as an ATP-binding catalytic site of the tyrosine kinase domain) and does not bind in a significant amount to other receptors present (e.g., off-target growth factor receptors). Specific binding or binding with high affinity can also mean, for example, that the binding compound, ligand, or binding composition binding to its target (e.g., a Syk inhibition) with an affinity that is often at least 20% greater, at least 25% greater, at least 50% greater, or at least 100% (2-fold) greater than the affinity of the compound, ligand, or binding composition to bind with another receptor (e.g., a growth factor receptor).

In a typical embodiment, a molecule that specifically binds a target will have an affinity that is at least about 10⁶ liters/mol (Ko=10^{˜6} M), and preferably at least about 10 liters/mol, as determined, for example, by Scatchard analysis. The binding specificity of the compound to a Syk receptor is determined using standard techniques known in the art and described herein.

When administered to a subject, the compound can inhibit Syk from phosphorylating an erythrocyte anion transporter Band 3 of the subject (e.g, a human). For example, the overall design of the compound can allow for the inhibition of Syk without disrupting other pathways (e.g., those regulated by growth factor kinases). While other Syk inhibitors are known, conventional Syk inhibitors are not specific to Syk (i.e., they inhibit several kinases in addition to Syk including, for example, growth factor receptors that are necessary for growth and development (e.g., a vascular endothelial growth factor receptor (VEGFR, such as VEGFR3) or other growth factor receptors)). Inhibition of growth factor kinases is typically not desirable as it can lead to dwarfism and other growth disorders in pediatric patients. For example, studies support that imatinib can delay vertical growth, hormone production, and vitamin D synthesis in a subject. Furthermore, upregulation (not inhibition) of these growth factors is often desirable in response to vaso-occlusion and the like as it can promote tissue repair and regeneration (e.g., revascularization of tissues).

Non-limiting examples of such growth factor receptors include hepatocyte growth factor receptor (HGFR), epidermal growth factor receptor (EGFR), fibroblast growth factor receptor 3 (FGFR3), and insulin-like growth factor 1 receptor (IGF1R).

HGFR (or MET) binds hepatocyte growth factor (HGF, also called scatter factor (SF)), transducing multiple biological processes such as cell motility, morphogenesis, proliferation, and survival. HGFR precursor is proteolytically cleaved, forming alpha and beta subunits that form disulfide linked heterodimers. HGFR is widely expressed in early development and deletion of the gene lethally disrupts embryogenesis. After kidney, liver, or heart injury, HGFR is upregulated to promote tissue repair and regeneration.

EGFR (or ErbB-1 or HER1) can bind a member of the epidermal growth factor (EGF) family of proteins. EGFR is a member of the ErbB family of receptors, which comprises EGFR (ErbB-1), Her2/neu (ErbB-2), Her3 (ErbB-3), and Her4 (ErbB-4). Upon activation by an EGF, EGFR undergoes a transition from an inactive monomeric form to an active homodimer. EGFR also can form a heterodimer with another ErbB receptor to form an active heterodimer. These receptors are involved in early development and participate in the renewal of stem cells in tissue such as skin, liver, gut, and mammary ducts.

FGFR3 binds members of the fibroblast growth factor (FGF) family of proteins. The FGF family is the largest family of growth factor receptor ligands and comprises 22 factors. Alternative splicing of four FGFR genes can result in the production of over 48 different isoforms of FGFR. Further, the FGFRs dimerize as hetero- and homo-dimers. FGFR3 regulates chondrocyte differentiation, proliferation, and apoptosis, vitamin D metabolism, osteogenesis, and postnatal mineralization of bone by osteoblasts, and is required for normal skeleton development and inner ear development.

IGF1R is activated by the hormone insulin-like growth factor 1 (IGF-1) and by a related hormone called IGF-2. IGF1R regulates prenatal and postnatal development, glucose metabolism, and neutrophil physiology, and promotes protective physiological hypertrophy in the left ventricle of the heart.

VEGFRs interact with vascular endothelial growth factors (VEGFs), which are signal proteins that stimulate the formation of blood vessels. There are three main subtypes of VEGFR—1, 2 and 3. Depending on alternative splicing, they can be membrane-bound or soluble. The VEGF family has five members, VEGF-A, placental growth factor (PGF), VEGF-B, BEGF-C, and VEGF-D. Alternative splicing leads to multiple isoforms. The lack of inhibition of VEGFRs, such as VEGFR3, is advantageous because the administration of the compound will not prevent the essential revascularization of tissues that become infarcted in a patient with sickle cell disease due to the occurrence of vaso-occlusive events.

In certain embodiments, the compound comprises a small molecule inhibitor has a specificity for Syk and inhibits Syk at least about 20 times better (e.g., 20 times better) than it inhibits a growth factor receptor (e.g., the small molecule inhibitor is Syk-specific).

Primarily, the docking scores for several of the compounds were assessed and are provided in Table 1. The determination of three-dimensional structures of protein-receptor complexes is an important part of structure-based drug design. A root mean square deviation of less than about 2 Å corresponds with good docking solutions.

TABLE 1
Docking Scores for Selected Compounds Against SYK Enzyme

| Compound | Docking Score (Å) |
|---|---|
| S970 | −10.63 |
| S1115 | −8.0 |
| S1213 | −8.6 |
| S1219 | −8.6 |
| S1214 | −7.75 |
| S2053 | −8.35 |
| S2054 | −8.45 |
| S1214 | −7.75 |

Additional surprising and unexpected properties of the compound (e.g., S970, S970D, S1115, S1213, 51219, S1213-CH₃, S2053, S2054, and S1214) are shown in Tables 2 and 3 as compared to commercially available/previously known Syk inhibitors. Activity (IC₅₀ nM) results for several selected compounds are shown in Table 2. The IC₅₀ value was determined as a concentration of the compound (or other tested Syk inhibitor) that reduced the identified receptor activity by 50%. Table 3 shows the selectivity ratio of each of the selected compounds as a ratio of the IC₅₀ Syk/kinases).

TABLE 2
Selectivity and Enzymatic Potency Data of Compounds vs. Commercially
Available Syk Inhibitors and FDA-Approved Syk Inhibitors

| | IC₅₀ (nM) | | | | | |
|---|---|---|---|---|---|---|
| Compound | Syk | HGFR | EGFR | FGFR3 | IGF1R | VEGFR |
| S970 | 177 | 40,000 | 8,000 | 10,000 | 10,000 | 4,000 |
| S970D | 80 | >40,000 | 650 | 6,000 | >5,000 | 2,100 |
| S1115 | 2.2 | >40,000 | 140 | 768 | 900 | 75 |
| S1213 | 25 | >20,000 | 25,000 | >3,000 | >20,000 | >2,000 |
| S1219 | 50 | | | | | |
| S1213-CH₃ | 113 | | | | | |
| S2053 | 147 | | 4,300 | >5,000 | >10,000 | >10,000 |
| S2054 | 680 | | | | | |
| S1214 | 530 | | | | | |

| | | | | | | |
|---|---|---|---|---|---|---|
| Inhibitor | Syk | HGFR | EGFR | FGFR3 | IGF1R | VEGFR |

Commercially Available Syk Inhibitors

| | | | | | | |
|---|---|---|---|---|---|---|
| Cerdulatinib | 32 | | | | | 51 |
| Entospletinib | 12 | 20,600 | 1,790 | 9,900 | 813 | 244 |
| Fostamtinib | 24.1 | 1,570 | 342 | 91.4 | 638 | 18.7 |
| Lanraplenib | 136 | >100,000 | 4,270 | 69,400 | 2,430 | 778 |
| Piceatannal | 3,440 | 35,000 | 17,600 | 24,700 | 21,500 | 5,000 |
| PRT062067 | 6.2 | 7,670 | 1,240 | 18,800 | 138 | 10,700 |
| Syk II | 70.3 | 69,400 | 27,000 | 36,900 | 13,500 | 4,210 |
| Syk IV | 69.3 | 1,260 | >100,000 | 321 | 460 | 91.2 |
| TAE-684 | 1,300 | 170 | 180 | 95 | 2.7 | 170 |
| TAK-659 | 8.5 | 3,160 | 27,600 | 19 | 4.500 | 150 |
| TAS05567 | 0.37 | | | | | |
| GSK143 | pIC₅₀ = 7.5 | | | | | |
| Syk-IN-1 | 31 | | | | | |
| Syk-IN-3 | 1 | | | | | |
| Syk-IN-4 | 0.31 | | | | | |
| SRX3207 | 10.7 | | | | | |
| RO9021 | 5.6 | | | | | |
| Gusacitinib | 5-46 | | | | | |
| R112 | 226 | | | | | |
| PRT-060318 | 4 | | | | | |
| OXSI-2 | 14 | | | | | |

FDA-approved Syk inhibitors for which Syk inhibition is known

| | | | | | | |
|---|---|---|---|---|---|---|
| Afatinib | >10,000 | 2,200 | 0.25 | >10,000 | >10,000 | >10,000 |
| (BIBW- | | | | | | |
| 2992) | | | | | | |
| Alectinib | 37,100 | 1,030 | 385 | 1,810 | 1,014 | 335 |
| Avapritinib | 280 | 25,000 | 8,730 | 1,015 | >100,000 | 2,580 |
| Axitinib | >10,000 | 820 | >10,000 | 210 | >10,000 | 170 |
| (AG-013736) | | | | | | |
| Bosutinib | 290 | 2,200 | 35 | >10,000 | >10,000 | >10,000 |
| (SKI-606) | | | | | | |
| Cerdulatinib | 32 | | | | | 51 |
| Crizotinib | 2,500 | 2.1 | >10,000 | 2,700 | 780 | >10,000 |
| Dasatinib | 2,900 | | 120 | 3,900 | | |
| Entrectinib | 244 | 8,270 | 2,650 | 246 | 80 | 441 |
| Erlotinib | >10,000 | 3,800 | 0.67 | >10,000 | >10,000 | 2,100 |
| Fedratinib | 2,700 | 8,500 | 3,400 | 360 | 2,200 | 2,000 |
| (TG101348) | | | | | | |
| Fostamtinib | 24.1 | 1,570 | 342 | 91.4 | 638 | 18.7 |
| Gefitinib | >10,000 | >10,000 | 1 | >10,000 | >10,000 | >10,000 |
| Imatinib | 31,000 | 11,200 | >10,000 | 26,700 | >100,000 | >10,000 |
| Lapatinib | >10,000 | >10,000 | 2.4 | >10,000 | >10,000 | >10,000 |
| Larotrectinib | 23,600 | >100,000 | 6,370 | 1,980 | >100,000 | 19,600 |
| Lestaurtinib | 9.5 | 290 | 1,100 | 580 | 4,200 | 17 |
| (CEP-701) | | | | | | |
| Midostaurin | 88 | 690 | 1,300 | 1,700 | 10,000 | 27 |
| (PKC412) | | | | | | |
| Neratinib | 5,100 | 4,000 | 1.1 | >10,000 | >10,000 | >10,000 |
| Nilotinib | >10,000 | >10,000 | >10,000 | >10,000 | >10,000 | >10,000 |
| Nintedanib | >10,000 | 200 | >10,000 | 93 | 62 | 95 |
| (BIBF-1120) | | | | | | |
| Pazopanib | 4,700 | 2,000 | >10,000 | 740 | >10,000 | 27 |
| Ripretinib | 13,200 | 3,020 | 9,930 | 84.4 | >100,000 | 0.24 |
| Ruxolitinib | >10,000 | >10,000 | >10,000 | >10,000 | >10,000 | >10,000 |
| (INCB18424) | | | | | | |
| Selumetinib | >10,000 | >10,000 | 7,000 | >10,000 | >10,000 | >10,000 |
| (AZD-6244/ | | | | | | |
| ARRY-886) | | | | | | |
| Sorafenib | >10,000 | >10,000 | >10,000 | >10,000 | >10,000 | 95 |
| Sunitinib | >10,000 | 7,200 | >10,000 | 290 | 2,600 | 50 |
| Tofcitinib | >10,000 | >10,000 | >10,000 | >10,000 | >10,000 | >10,000 |
| (CP-690550) | | | | | | |
| Tucatinib | 4,930 | 11,100 | 38.8 | 66,500 | >100,000 | 1,960 |
| Vandetanib | >10,000 | 5,700 | 9.5 | 1,600 | >10,000 | 1,100 |
| (ZD-6474) | | | | | | |

TABLE 3
Selectivity Results
Ratio of IC₅₀ SYK/Kinases

| Compound | SYK | VEGFR3 | EGFR | HGFR | FGF3R | IGF1R |
|---|---|---|---|---|---|---|
| S970 | 1 | 22 | 44 | 220 | 55 | 55 |
| S970D | 1 | 26 | 8 | >500 | 75 | >62 |
| S1115 | 1 | 34 | 63 | >18180 | 272 | 409 |

As shown in Tables 2 and 3, the following FDA-approved inhibitors are known to inhibit growth factor receptor kinases (i.e., HGR, EGFR, VEGFR, EGFR, IGF1R, and FGFR3): afatinib, almonertinib, anlotinib, axitinib, brigatinib, bosutinib, cabozantinib, capmatinib, cerdulatinib, ceritinib, crizaotinib, dacomitinib, dasatinib, erdafitinib, erlotinib, fostamtinib, fruquintinib, gefitinib, icotinib, lapatinib, Lenvatinib, lestaurtinib, lorlatinib, midostaurin, neratinib, nintedanib, Osimertinib, pemigatinib, pralsetinib, pyrotinib, regorafenib, selpercatinib, sorafenib, tepotinib, and vandetanib. None of the following inhibitors were more than 20-fold more potent for Syk compared to growth factor receptor kinases: alectinib, avapritinib, entrectinib, fedratinib, imatinib, Larotrectinib, nilotinib, pazopanib, ripretinib, ruxolitinib, selumetinib, sunitinib, tofacitinib, and tucatinib. Of the ten commercially available Syk inhibitors, namely, ceerdulatinib, entospletinib, fostamtinib, lanraplenib, piceatannal, PRT062067, Syk II, Syk IV, TAE-684, and TAK-659, only entospletinib, PRT062067, and Syk II were at least 20-fold more potent for Syk than for growth factor receptor kinases.

Furthermore, the cellular potency data of the compounds as compared to selected commercially available Syk inhibitors and FDA-approved inhibitors for which Syk inhibition is known are shown in Table 4. The half maximal effective concentration (EC₅₀) represents the concentration of a drug that is necessary to cause half of the maximum possible effect after a specified exposure.

TABLE 4
Inhibition of Band 3 Tyrosine Phosphorylation
EC₅₀ (nM)

| | pTyr | pTyr8 | pTyr359 |
|---|---|---|---|
| Compound | | | |
| S970 | 1,500 | 4,400 | 10,000 |
| S970D | 716 | 572 | |
| S1115 | 226 | 262 | 1,590 |
| S1213 | 1,500 | NP | NP |
| Inhibitor | | | |
| Entosplentinib | 3,000 | | |
| PRT062067 | 300 | | |
| Lanraplenib | 3,000 | | |
| Imatinib | 5,000 | | |
| R406 | 5,000 | | |

The above compounds can be synthesized in accordance with methods known in the art and exemplified herein. See, e.g., Examples 1 and 3-5.

Solubility of selected compounds was also tested using methods known in the art, with resulting data shown in Table 5.

TABLE 5
CLogP Data

| Compound | LogP | CLogP |
|---|---|---|
| S970 | 2.29 | 3.97 |
| S970D | 2.28 | 3.46 |
| S1115 | 1.42 | 3.15 |
| S1213 | 1.04 | 3.20 |
| S1219 | 1.54 | 1.18 |
| S2053 | 2.63 | 3.69 |

The compound can have a CLogP value between about 2.0 and about 5.0 (e.g., between 2.0 and about 5.0, about 2.0 and 5.0, or 2.0 and 5.0). In certain embodiments, e.g., compound S1213, the compound has a CLogP value of between about 1.0 and about 2.0 (e.g., between 1.0 and about 2.0, about 1.0 and 2.0, or 1.0 and 2.0). The term “cLogP” refers to a calculated partition coefficient which in the physical sciences, is the ratio of concentrations of a compound in a mixture of the two immiscible phases at equilibrium. These coefficients are a measure of the difference in solubility of the compound in these two phases. In general, this is a measure of hydrophobicity of the compound as one phase is aqueous (traditionally water) and the other is hydrophobic (traditionally octanol). Higher values indicate greater partitioning of the compound into the hydrophobic phase and support increased hydrophobicity.

In certain embodiments, the compound has a polar surface area of less than about 250 square Å (e.g., less than 250 square Å). In certain embodiments, the compound has no more than 5 H-bond donors. In certain embodiments, the compound has no more than 15 H-bond acceptors. In certain embodiments, the compound has no more than 20 rotatable bonds. In certain embodiments, the compound can exhibit more than one of the aforementioned properties.

The compounds hereof also exhibit sufficient oral bioavailability. Table 6 shows data of selected compounds related to the absolute oral bioavailability thereof (studies performed using methods commonly known in the art).

TABLE 6
Absolute Oral Bioavailability Data

| Compound | F |
|---|---|
| S970 | NP |
| S970D | NP |
| S1115 | 2% |
| S1213 | 2% |
| S1219 | 1.1% |
| S2053 | 8% |

Administration of the compounds, pharmaceutically acceptable salts, or compositions hereof can inhibit Syk from phosphorylating an arythrocyte anion transporter Band 3. The compounds or pharmaceutically acceptable salts thereof are at least 20-fold more potent for Syk than for growth factor receptor kinases; in other words, the compounds have a higher affinity for Syk as compared to conventional Syk inhibitors and such affinity is discriminatory, meaning the compounds hereof do not share this high affinity with other receptors, such as growth factor receptor kinases.

The compounds (or pharmaceutically acceptable salts thereof) can contain one or more chiral centers or may otherwise exist as multiple stereoisomers, such as enantiomers, diastereomers, and enantiomerically or diastereomerically enriched mixtures. Unless stated otherwise, it is intended that all stereoisomeric forms of the compounds are contemplated. When the compounds contain alkene double bonds, and unless specified otherwise, it is intended that this includes both E and Z geometric isomers (e.g., cis or trans). Likewise, all possible isomers, as well as their racemic and optically pure forms, and all tautomeric forms are also intended to be included. One of ordinary skill in the art will further appreciate that the compounds can be “deuterated,” meaning one or more hydrogen atoms can be replaced with deuterium.

The compounds (or pharmaceutically acceptable salts thereof) can exist in un-solvated forms as well as solvated forms, including hydrated forms. In general, the solvated forms are equivalent to un-solvated forms. The compounds can exist in multiple crystalline or amorphous forms. In general, all physical forms are equivalent for the uses contemplated. The formulae include pharmaceutically acceptable salts (e.g., acid addition and base salts), hydrates, and/or solvates.

### Pharmaceutically Acceptable Salts

“Pharmaceutically acceptable salts” of the compounds are contemplated. The term “pharmaceutically acceptable salt” refers to those salts whose counter ions can be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases also can be formed, for example, hemisulphate and hemicalcium salts.

### Pharmaceutical Compositions, Routes of Administration, and Dosing

Further provided is a pharmaceutical composition comprising an above-described compound and a pharmaceutically acceptable carrier or excipient. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The carrier can be an excipient. The choice of carrier can depend on factors such as the particular mode of administration, the effect of the carrier on solubility and stability, and the nature of the dosage form. Pharmaceutical compositions suitable for the delivery of compounds as described herein and methods for their preparation may be found, for example, in Remington: The Science & Practice of Pharmacy, 21st edition (Lippincott Williams & Wilkins, 2005).

The components of the compositions also can be commingled with the compound, and with each other, in a manner such that there is no interaction which would substantially impair the desired pharmaceutical efficiency.

The composition can comprise cremophor, polysorbate, nanoparticles, a polymer, or a hydrogel, for example. In certain embodiments, the pharmaceutical composition comprises a plurality of compounds and a pharmaceutically acceptable carrier. In certain embodiments, a pharmaceutical composition further comprises at least one additional pharmaceutically active agent. The at least one additional pharmaceutically active agent can be an agent useful in the treatment of ischemia-reperfusion injury.

Pharmaceutical compositions can be prepared by combining one or more compounds with a pharmaceutically acceptable carrier and, optionally, one or more additional ingredients (e.g., pharmaceutically active ingredients). The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

A pharmaceutically acceptable carrier can include any and all solvents, dispersion media, coatings, antibacterial and antifungal agents, isotonic and absorption delaying agents, and the like, and combinations thereof, that are physiologically compatible. The carrier can be suitable for parenteral administration. Pharmaceutically acceptable carriers include sterile aqueous solutions or dispersions and sterile powders for the extemporaneous preparation of sterile injectable solutions or dispersions. Examples of such carriers (or excipients) include, but are not limited to, calcium carbonate, calcium phosphate, various sugars, starches, cellulose derivatives, gelatin, and polymers such as polyethylene glycols. One or more other active agents also can be incorporated into a pharmaceutical composition.

The pharmaceutical composition can be formulated as a liquid, e.g., a suspension or a solution A liquid formulation can comprise water, ethanol, polyethylene glycol, propylene glycol, methylcellulose, or a suitable oil, and one or more emulsifying agents and/or suspending agents. A liquid formulation can be prepared by the reconstitution of a solid.

Pharmaceutical formulations (e.g., for parenteral administration) include aqueous solutions of the active compounds in water-soluble form. Additionally, suspensions of the active compounds can be prepared as appropriate oily injection suspensions. An aqueous suspension can contain a compound, alone or in further combination with one or more other active agents, in admixture with an appropriate excipient. Excipients include suspending agents, such as sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide, e.g., lecithin; a condensation product of an alkylene oxide with a fatty acid, e.g., polyoxyethylene stearate; a condensation product of ethylene oxide with a long-chain aliphatic alcohol, e.g., heptadecaethyleneoxycetanol; a condensation product of ethylene oxide with a partial ester derived from fatty acids and a hexitol, such as polyoxyethylene sorbitol monooleate; or a condensation product of ethylene oxide with a partial ester derived from fatty acids and hexitol anhydrides, e.g, polyoxyethylene sorbitan monooleate. The aqueous suspension also can contain one or more preservatives, e.g., ascorbic acid or ethyl, n-propyl, or p-hydroxybenzoate, and one or more coloring agents. In certain embodiments, an aqueous suspension can further comprise suitable lipophilic solvents or vehicles including fatty oils such as sesame oil, or synthetic fatty acid esters, such as ethyl oleate or triglycerides, or liposomes. Optionally, the suspension can also contain suitable stabilizers or agents that increase the solubility of the compounds to allow for the preparation of highly concentrated solutions.

Alternatively, the pharmaceutical compositions can be in powder form for constitution with a suitable vehicle, e.g., sterile pyrogen-free water, before use. Dispersible powders and granules suitable for preparation of an aqueous suspension by the addition of water can provide the active ingredient in admixture with a suspending agent, a dispersing or wetting agent, and one or more preservatives. Additional excipients, for example, coloring agents, also can be present.

Suitable emulsifying agents include naturally occurring gums, e.g., gum acacia or gum tragacanth; naturally occurring phosphatides, e.g., soybean lecithin; and esters, including partial esters derived from fatty acids and hexitol anhydrides, e.g., sorbitan mono-oleate, and condensation products of partial esters with ethylene oxide, e.g., polyoxyethylene sorbitan monooleate. Isotonic agents, e.g., sugars, polyalcohols, such as mannitol, sorbitol, or sodium chloride, can be included in the composition. Prolonged absorption of injectable compositions can be achieved by including in the composition one or more agents to delay absorption, e.g., monostearate salts and gelatin.

For use in therapy or treatment, an effective amount of the compound or composition can be administered to a subject by any mode that delivers the compound as desired. Administering a composition can be accomplished by any means known to the skilled artisan. Routes of administration include, but are not limited to, intravenous, intramuscular, intraperitoneal, intravesical (urinary bladder), oral, subcutaneous, direct injection, mucosal (e.g., topical to eye), inhalation, and topical.

Colorants and/or flavoring agents can be included. For example, the compound can be formulated (such as by liposome or microsphere encapsulation) and then further contained within an edible product, such as a refrigerated beverage containing colorants and flavoring agents.

Illustrative formats for oral administration include, but are not limited to, tablets, capsules, elixirs, syrups, and the like.

In certain embodiments, a compound can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular, intranasal, and subcutaneous. Suitable means for parenteral administration include needle (including microneedle) injectors, needle-free injectors, and infusion techniques. Where it is desirable to deliver the compound(s) and/or compositions systemically, the compound(s) and/or composition can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Parenteral formulations are typically aqueous solutions that can contain carriers or excipients, such as salts, carbohydrates, and buffering agents (preferably at a pH of 3-9), but, for some applications, they may be more suitably formulated as a sterile non-aqueous solution or as a dried form to be used in conjunction with a suitable vehicle, such as sterile, pyrogen-free water.

A liquid formulation can be adapted for parenteral administration of a compound. The preparation of parenteral formulations under sterile conditions, for example, by lyophilization under sterile conditions, can readily be accomplished using standard pharmaceutical techniques well-known to those skilled in the art. The solubility of a compound can be increased by the use of appropriate formulation techniques, such as the incorporation of solubility-enhancing agents.

Formulations for parenteral administration can be formulated for immediate and/or modified release. A compound can be administered in a time-release formulation, for example in a composition which includes a slow-release polymer. The compound can be prepared with a carrier that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

Sterile injectable solutions can be prepared by incorporating the compound(s), alone or in further combination with one or more other active agents, in the required amount in an appropriate solvent with one or a combination of ingredients described above, as required, followed by filtered sterilization. Typically, dispersions are prepared by incorporating the compound into a sterile vehicle, which contains a dispersion medium and any additional ingredients of those described above. In the case of sterile powders for the preparation of sterile injectable solutions, the preferred methods of preparation are vacuum-drying and freeze-drying, which yield a powder of the active ingredients plus any additional desired ingredient from a previously sterile-filtered solution thereof, or the ingredients can be sterile-filtered together.

The pharmaceutical composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion, and by the use of surfactants.

A compound, or a pharmaceutical composition comprising a compound, can be continuously administered, where appropriate.

### Methods of Treatment

Methods of treating RBC-mediated diseases (e.g., SCD) are provided. The method can comprise administering to a subject (e.g., a subject experiencing or at risk of experiencing an RBC-mediated disease) an effective amount of an above-described compound, a pharmaceutically acceptable salt thereof, or any of the above-described pharmaceutical compositions. In certain embodiments, the compound is of formula (X):

A-B-C (X)

or is a pharmaceutically acceptable salt thereof, wherein:
A is a first ring that is nitrogen-linked and optionally substituted, the first ring comprising a fused bicyclic ring or a monocyclic ring with each ring each first ring comprising a 4-6-membered heterocycle or carbocycle;
B is a second ring, optionally substituted with at least one substituent, the second ring comprising a monocyclic ring or a fused bicyclic or tricyclic ring, and wherein each ring of the second ring comprises a 5- or 6-membered heterocycle or carbocycle; and
C is a third bicyclic ring comprising a 5- or 6-membered carbocycle fused with a 5- or 6-membered carbocycle or heterocycle, wherein the third bicyclic ring is substituted with at least a first substituent comprising:

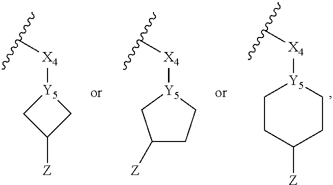

or an amine, or an alcohol,
   wherein:
      X₄ is each independently CH₂, NH, CO, O, or S,
      Y₅ is each independently CH or N,
         Z is selected from OH, SH, NIH, CO₂H, NHCH₃, and

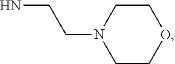

and
[symbol] is a point of attachment of the first substituent to the third bicyclic ring;
   wherein the compound or pharmaceutically acceptable salt thereof inhibits Syk tyrosine kinase at a concentration that is at least 20-fold lower than the concentration at which the compound (or pharmaceutically acceptable salt thereof) inhibits HGFR, EGFR, FGFR3, IGF1R, and/or a VTGFR.

The method can further comprise administering to the subject an effective amount of at least one additional/other active agent (each a “second active agent”). In certain embodiments, the second active agent is selected from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, Syk-IN-1, SRX3207, R09021, gusacitinib, R112, PRT-060318, and OXSI-2. Additionally or alternatively, the method can comprise administering an effective amount of: (a) an above-described compound, a pharmaceutically acceptable salt thereof, or any of the above-described pharmaceutical compositions; (b) fostamatinib; (c) PRT062607; (d) TAK-659; (e) TAE-684; (f) entospletinib; (g) lanraplenib; (h) cerdulatinib; (i) piceatannol; (j) S701; (k) Syk II; (1) Syk IV; (m) TAS05567; (n) GSK143; (o) Syk-IN-3; (p) Syk-IN-4; (q) Syk-IN-1; (r) SRX3207; (s) R09021; (t) gusacitinib; (u) 112; (v) PRT-060318; (w) OXSI-2; (x) (a) and (b); (y) (a) and (c); (z) (a) and (d); (aa) (a) and (e); (ab) (a) and (f); (ac) (a) and (g); (ad) (a) and (h); (ae) (a) and (i); (af) (a) and (j); (ag) (a) and (k), (ah) (a) and (l); (ai) (a) and (m); (aj) (a) and (n); (ak) (a) and (o); (al) (a) and (p); (am) (a) and (q); (an) (a) and (r); (ao) (a) and (s); (ap) (a) and (t); (aq) (a) and (u); (ar) (a) and (v); (as) (a) and (w); or (at) (a) and one or more of (b)-(w). The aforementioned can optionally be administered to a subject as a single pharmaceutical composition comprising a pharmaceutically acceptable carrier or excipient, or in multiple dosages using, for example, multiple pharmaceutical compositions.

The at least one second active agent can comprise a combination of two or more of fostamatinib, PRT062607, TAK-659, TAE-684, entospletinib, lanraplenib, cerdulatinib, piceatannol, 5701, Syk II, Syk IV, TAS05567, GSK143, Syk-IN-3, Syk-IN-4, Syk-IN-1, SRX3207, RO9021, gusacitinib, R112, PRT-060318, and OXSI-2. Each second active agent can be administered simultaneously or sequentially, and in either order (where applicable), with the compound, pharmaceutically acceptable salt thereof or pharmaceutical composition. In certain embodiments, the compound, pharmaceutically acceptable salt thereof or pharmaceutical composition and each second active agent are administered by the same or different routes.

The method can further comprise administering to the subject an effective amount of hydroxyurea (which can, for example, comprise a second active agent). The hydroxyurea can be administered simultaneously or sequentially, and in either order (where applicable), with the compound, pharmaceutically acceptable salt thereof or pharmaceutical composition. In certain embodiments, the compound, pharmaceutically acceptable salt thereof or pharmaceutical composition and the hydroxyurea are administered by the same route. In certain embodiments, the compound, pharmaceutically acceptable salt thereof or pharmaceutical composition and the hydroxyurea are administered by different routes.

Another method of treating an RBC-mediated disease is also provided. In certain embodiments, the method comprises administering an effective amount of a compound (or pharmaceutically acceptable salt):
(i) which comprises a rigid, fused bicyclic ring, which is substituted with at least two substituents, at least one of which is an N-linked, optionally substituted, carbo- or heterocycle and the other of which is an indole substituted with at least one substituent, at least one of which is a 4-6-membered, optionally substituted, carbo- or heterocycle,
(ii) which inhibits Syk from phosphorylating the human erythrocyte anion transporter band 3, and
(iii) which inhibits Syk tyrosine kinase at a concentration that is at least about 20-fold lower than the concentration at which the compound inhibits HGFR, EGFR, FGFR3, IGF1R, and/or a VEGFR,
wherein, additionally or alternatively to (ii) and (iii), the compound or pharmaceutically acceptable salt of (i) has:
(iv) a CLogP value between about 2 and about 5 (e.g. between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds,
   where upon the subject is treated for sickle cell disease. The compound can inhibit Syk from phosphorylating tyrosine 8 and tyrosine 21 in the human erythrocyte anion transporter band 3. The compound and/or pharmaceutical composition comprising the compound can stabilize erythrocyte cell membranes and reduce the release of microvesicles from sickle cells.

The RBC-mediated disease can be selected from the group consisting of SCD, thalassemia (e.g., α-thalassemia or β-thalassemia), G6PD, glutathione reductase deficiency, and a disease involving the release of microvesicles from blood cells. Each of these diseases are related to the phosphorylation of erythrocyte anion transporter band 3, and inhibition of such phosphorylation can prevent, slow, and/or treat the same such that the sequelae that cause vaso-occlusion and disease symptoms is either not initiated or halted. Indeed, administration of an effective amount of a compound or pharmaceutical composition hereof can stabilize erythrocyte cell membranes in the subject and reduce the release of hemoglobin from sickle cells in the subject. In this manner, the risk of vaso-occlusive crisis in the subject can be reduced.

In certain embodiments of the method, administration of the compound, pharmaceutically acceptable salt thereof, or pharmaceutical composition inhibits Syk tyrosine kinase at an IC₅₀ of 200 nM or lower. The subject can be a human child (i.e., a growing or other human where off-target inhibition of a growth hormone could have significant deleterious effect).

The method can further comprise the simultaneous or sequential administration, in either order, of one or more other compounds selected from the group consisting of fostamatinib, PRT062607, TAK-659, TAE-684, entospletinib, lanraplenib, cerdulatinib, piceatannol, 5701, Syk II, Syk IV, TAS05567, GSK143, Syk-IN-3, Syk-IN-4, SRX3207, R09021, gusacitinib, R112, PRT-060318, and OXSI-2, optionally as a pharmaceutical composition comprising a pharmaceutically acceptable carrier or excipient. The one or more other compounds can be administered by the same or different routes (as compared to administration of the compound or pharmaceutical composition).

Methods of preventing or inhibiting the phosphorylation of human erythrocyte anion transporter band 3 in a subject comprising administering to the subject an effective amount of an above-described compound, a pharmaceutically acceptable salt thereof, or any of the above-described pharmaceutical compositions.

Still further provided is a method of treating a subject for a disease involving the release of microvesicles from blood cells. The method comprises administering an effective amount of a compound or a pharmaceutically acceptable salt thereof, wherein the compound comprises a rigid, fused bicyclic ring, which is substituted with at least two substituents, at least one of which is an N-linked, optionally substituted, carbo- or heterocycle and the other of which is an indole substituted with at least one substituent that is a 4-6-membered, optionally substituted, carbo- or heterocycle. Upon administration to the subject, the subject can be treated for the disease involving the release of microvesicles. The disease can be thalassemia. The thalassemia can be α-thalassemia or β-thalassemia, alone or in combination with SCID. The disease can be G6PD deficiency. The disease can be glutathione reductase deficiency.

The method can further comprise the simultaneous or sequential administration (as compared to the compound or pharmaceutical composition hereof), in either order of, a Src inhibitor, optionally as a pharmaceutical composition comprising a pharmaceutically acceptable carrier or excipient. The Src inhibitor can be administered by the same or different routes as compared to the compound or pharmaceutical composition hereof. The Src inhibitor can be dasatinib, ibrutinib, bafetinib, PP1, PP2, PP121, or a combination of two or more of the foregoing.

Even still further provided is a method of treating SCD in a subject, which method comprises administering an effective amount of a compound:
(i) which comprises a pyrazine, which is substituted with at least three substituents, at least one of which is a carboxamide or a lactam, another of which is an N-linked, optionally substituted, carbo- or heterocycle, and another of which is a carbo- or heterocycle with at least one substituent, at least one of which is a 4-6-membered, optionally substituted, carbo- or heterocycle,
(ii) which inhibits Syk tyrosine kinase from phosphorylating the human erythrocyte anion transporter band 3,
(iii) which inhibits Syk tyrosine kinase at a concentration that is at least about 20-fold lower than the concentration at which the compound inhibits HGFR, EGFR, FGFR3, IGF1R, and/or a VEGFR,
wherein, additionally or alternatively to (ii) and (iii), the compound of (i) has:
(iv) a CLogP value between about 2 and about 5 (e.g., between 2 and about 5, about 2 and 5, or 2 and 5),
(v) a polar surface area of less than about 250 square Å (e.g., less than 250 square Å),
(vi) no more than 5 H-bond donors,
(vii) no more than 15 H-bond acceptors, and/or
(viii) no more than 20 rotatable bonds,
where upon the subject is treated for SCD. The compound or pharmaceutically acceptable salt thereof can inhibit Syk tyrosine kinase from phosphorylating tyrosine S and tyrosine 21 in the human erythrocyte anion transporter band 3. The compound or pharmaceutically acceptable salt thereof can stabilize erythrocyte cell membranes and reduce the release of microvesicles from sickle cells. The compound or pharmaceutically acceptable salt thereof can stabilize erythrocyte cell membranes and reduce the release of hemoglobin from sickle cells. The risk of Vaso-occlusive crisis in the subject can be reduced. The compound or pharmaceutically acceptable salt thereof can inhibit Syk tyrosine kinase at an IC₅₀ of 200 nM or lower. The subject can be a human child. The method can further comprise the simultaneous or sequential administration, in either order, of one or more of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, SRX3207, R09021, gusacitinib, R112, PRT-060318, and OXSI-2, optionally as a pharmaceutical composition comprising a pharmaceutically acceptable carrier or excipient. The one or more other compounds can be administered by the same or different routes.

A method of treating a subject for a disease involving the release of microvesicles from blood cells is also provided. The method comprises administering an effective amount of a compound or pharmaceutically acceptable salt thereof, wherein the compound comprises a pyrazine, which is substituted with at least three substituents, at least one of which is a carboxamide or a lactam, another of which is an N-linked, optionally substituted, carbo- or heterocycle, and another of which is a carbo- or heterocycle with at least one substituent, at least one of which is a 4-6—, membered, optionally substituted, carbo- or heterocycle, whereupon the subject is treated for the disease involving the release of microvesicles. The disease can be thalassemia. The thalassemia can be α-thalassemia or β-thalassemia, alone or in combination with sickle cell disease. The disease can be G6PD deficiency. The disease can be glutathione reductase deficiency. The method can further comprise the simultaneous or sequential administration, in either order of, a Src inhibitor, optionally as a pharmaceutical composition comprising a pharmaceutically acceptable carrier or excipient. The Src inhibitor can be administered by the same or different route as compared to the compound or pharmaceutically acceptable salt thereof. The Src inhibitor can be dasatinib, ibrutinib, bafetinib, PP1, PP2, PP121, or a combination of two or more of the foregoing.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

As used herein, an “effective amount” refers to any amount of a compound with respect to use in treatment, refers to an amount of the compound in a preparation which, when administered as part of a desired dosage regimen (to a mammal, such as a human) that is sufficient to achieve a desired biological effect (e.g., alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment).

Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation.

For example, for any compound, effective amount can be initially determined from animal models. An effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug.

“Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds or pharmaceutically acceptable salts thereof can be administered to human subjects in effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound or pharmaceutically acceptable salt thereof.

The compounds or pharmaceutically acceptable salts thereof can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds or pharmaceutically acceptable salts thereof can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 m/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound, pharmaceutically acceptable salt thereof or precursor thereof can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 150 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound, or pharmaceutically acceptable salt thereof, or precursor thereof can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g, topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., ethylenediaminetetraacetic acid (EDTA) for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochen 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof or pharmaceutically acceptable salt thereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound or a pharmaceutically acceptable salt thereof beyond the stomach environment, such as in the intestine.

To ensure full gastric resistance a coating impermeable to at least pH 5.0 can be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

As noted above, the compound or pharmaceutically acceptable salt thereof can be included in the formulation as fine multi-particulates in the form of granules or pellets of particle size about 1 mm. The formulation of the material for capsule administration could also be as a powder, lightly compressed plugs or even as tablets.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colorado, March, (recombinant hepatocyte growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g, sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

Disintegrants can be included in the formulation of therapeutic agent into a solid dosage form Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold the compound together to form a hard tablet and include materials from natural products such as acacia, tragacanth, starch and gelatin. Others include methyl cellulose (MCI), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, tale, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

One may dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride, Some commercially available diluents are Fast-Flo, Emdex, STA-Rx 1500, Emcompress and Avicell.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

Therapeutic agent(s), including specifically, but not limited to, a compound, can be provided in particles. “Particles” as used herein means nanoparticles or microparticles (or in some instances larger particles) that can consist in whole or in part of the compound or the other therapeutic agent(s) as described herein. The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:581-587 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Therapeutic agent(s) can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

Depending upon the route of administration, a wide range of permissible dosages are contemplated herein, including doses falling in the range from about 10⁶ to 10¹¹ virus particles (VP)/kg. The dosages may be single or divided and may be administered according to a wide variety of protocols, including q.d. (once a day), b.i.d. (twice a day), t.i.d. (three times a day), or even every other day, once a week, once a month, once a quarter, and the like. In each of these cases it is understood that the effective amounts described herein correspond to the instance of administration, or alternatively to the total daily, weekly, month, or quarterly dose, as determined by the dosing protocol.

In addition to the illustrative dosages and dosing protocols described herein, an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

Those skilled in the art will recognize that numerous modifications can be made to the specific implementations described above. The implementations should not be limited to the particular limitations described. Other implementations may be possible.

While the compounds and pharmaceutical compositions have been illustrated and described in detail in the foregoing description, the same is to be considered as illustrative and not restrictive in character, it being understood that only certain embodiments have been shown and described and that all changes and modifications that come within the spirit of the invention are desired to be protected.

It is intended that that the scope of the present compounds, compositions, and methods are defined by the following claims. However, this disclosure may be practiced otherwise than is specifically explained and illustrated without departing from its spirit or scope. Those skilled in the art will understand that various alternatives to the embodiments described herein can be employed in practicing the claims without departing from the spirit and scope as defined in the following claims.

Any use of section headings is intended to aid reading of the document and is not to be interpreted as limiting. Further, information that is relevant to a section heading may occur within or outside of that particular section.

All publications, patents, patent application publications, journal articles, textbooks, and other publications referred to in this document are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### Certain Definitions

As used herein, the following terms and phrases shall have the meanings set forth below. Unless defined otherwise, all technical and scientific terms used herein have the same meaning as commonly understood to one of ordinary skill in the art.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. Thus, for example, references to “the method” includes one or more methods and/or steps of the type, which are described herein and/or which will become apparent to those ordinarily skilled in the art upon reading the disclosure.

The term “or” is used to refer to a nonexclusive “or” unless otherwise indicated. In addition, it is to be understood that the phraseology or terminology employed herein, and not otherwise defined, is for the purpose of description only and not of limitation.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O-alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Arylalkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

The term “heteroalkyl” refers to an alkyl group as defined above in which one or more skeletal carbon atoms of the alkyl are substituted with a heteroatom (with the appropriate number of substituents or valences—for example, —CH₂— can be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

The terms “protein,” “polypeptide” and “peptide” refer to compounds comprising amino acids joined via peptide bonds and are used interchangeably.

### EXAMPLES

The following examples serve to illustrate the present disclosure. The examples are not intended to limit the scope of the claims in any way.

### Example 1

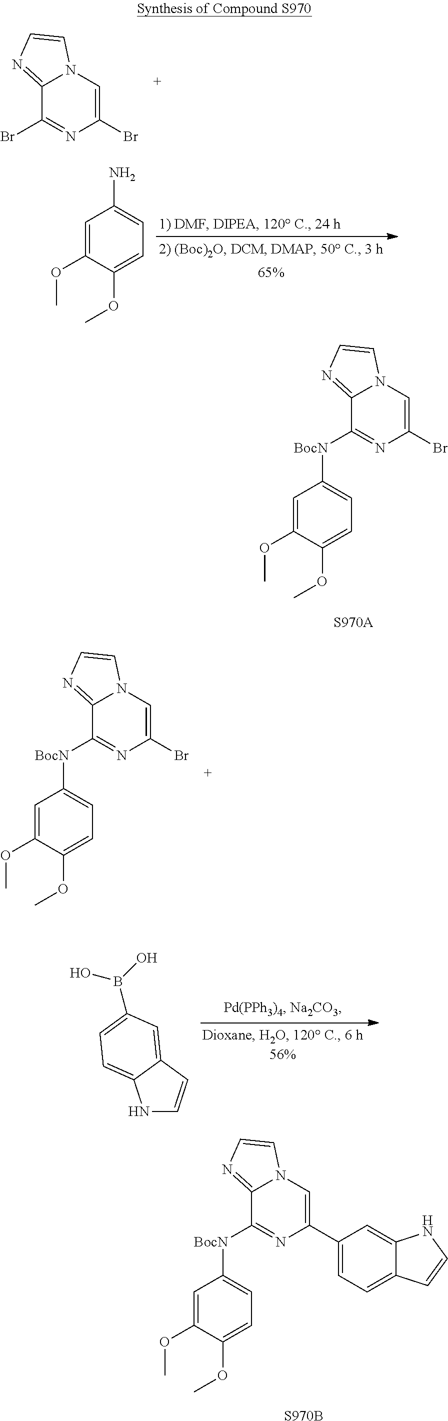

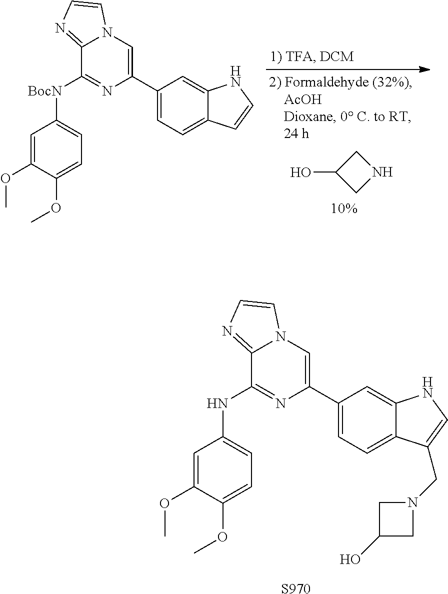 [SMILES: COc1ccc(N(C(=O)OC(C)(C)C)c2nc(-c3ccc4cc[nH]c4c3)cn3ccnc23)cc1OC ; OC1CNC1 ; COc1ccc(Nc2nc(-c3ccc4c(CN5CC(O)C5)c[nH]c4c3)cn3ccnc23)cc1OC ; "S970"]

tert-butyl (6-bromoimidazo [1,2-a]pyrazin-8-yl)(3,4-dimethoxyphenyl)carbamate (Compound S970-A). N,N-Diisopropylethylamine (DIPEA) (2.5 mL, 5.0 eqiv) was added to a stirred solution of 6,8-dibromoimidazo[1,2-a]pyrazine (1.0 g 3.6 mmol) and 3,4-dimethoxyaniline (426 mg, 2.5 mmol) in dimethylformamide (DMF) (20.0 mL). The reaction mixture was heated to 120° C. and stirred at same temperature for 24 hours. After the reaction was completed (progress of the reaction was monitored by thin-layer chromatography (TLC))), it was allowed to cool to room temperature and then added to 250 ml of ice-cold water. The resulting precipitates were stirred at room temperature for 30-45 minutes and filtered through the Buchner funnel. The resulting wet material was washed with water (100 mL) followed by diethyl ether (100 mL) and dried under vacuum at 50° C. for 5 hours to afford the 6-bromo-N-(3,4-dimethoxyphenyl)imidazo[1,2-a]pyrazin-8-amine as a solid.

The resulting crude intermediate was charged into 100 ml single neck RBF and added 25 ml dichloromethane (DCM) followed by (Boc)₂O anhydride (0.875 g, 4.0 mmol) and catalytic amount of 4-Dimethylaminopyridine (DMAP) (25 mg, 0.025 equiv). The resulting solution was heated to 50° C. and stirred for 3 hours. After completion of the reaction (progress of the reaction was monitored by TLC), the resulting solution was concentrated to dry under vacuum.

The resulting crude material was purified by normal phase silica gel chromatography (Hexanes: EtOAc) to give an off-white solid (1.00 g, 65% overall yield).

¹H NMR (500 MHz, dimethyl sulfoxide (DMSO)-d6): δ 8.89 (s, 1H), 8.13 (s, 1H), 7.86 (s, 1H), 6.99-6.82 (m, 2H), 6.74 (d, J=8.5 Hz, 1H), 3.71 (s, 3H), 3.68 (s, 3H), 1.29 (s, 9H) ppm; ¹³C NMR (125 MHz, DMSO-d6): δ 152.9, 149.0, 148.1, 145.4, 136.9, 136.6, 133.6, 120.7, 119.7, 119.0, 116.8, 111.9, 111.8, 81.9, 56.1, 56.0, 28.0 ppm. LCMS (ESI) m/z calcd for C₁₉H₂₂BrN₄O₄: 449.0819 Found: 449.0817 (M+1)⁺.

tert-butyl (6-(1H-indol-6-yl)imidazo[1,2-a]pyrazin-8-yl)(3,4-dimethoxyphenyl)carbamate (Compound 5970-B).

Compound S970-A (500 mg, 1.11 mmol) and (1H-indol-5-yl)boronic acid (220 mg, 1.35 mmol) were dissolved in 1,4 Dioxane (8 ml) followed by the addition of 120 (4 in) in a vial fitted with a screw cap with a septa. The reaction mixture was purged with a gentle stream of N₂ with stirring. After a 5 minute purge, the exit needle was removed and added Pd(PPh3)₄(40 mg, 0.025 mmol) followed quickly by Na₂CO₃ (356 mg, 3.35 mmol). The reaction mixture was heated to 120° C. and stirred at same temperature for 6 hours. The reaction mixture was then concentrated to dry and the desired product was isolated by normal phase silica gel chromatography (Hexanes: EtOAc) to give off-white solid (302 mg, 56%).

¹H NMR (500 MHz, DMSO-d6): δ 11.25 (S, 1H), 9.11 (s, 111), 8.13 (s, 111), 8.00 (s, 1H), 7.80 (s, 1H), 7.68-7.50 (m, 2H), 7.42-7.30 (m, 1H), 7.14-7.03 (m, 1H), 6.96-6.77 (m, 2H), 6.44 (s, 1H), 3.72 (s, 3H), 3.70 (s, 3H) ppm; ¹³C NMR (125 MHz, DMSO-d6): δ 153.3, 148.9, 147.9, 145.5, 137.9, 136.9, 136.7, 135.6, 134.0, 129.0, 128.4, 127.2, 120.8, 119.6, 117.2, 116.5, 115.5, 111.95, 111.92, 109.5, 101.6, 81.3, 561, 56.0, 28.1 ppm. LCMS (ESI) m/z calcd for C₂₇H₂₈N₅O₄: 486.2136 Found: 486.2133 (M+1)⁺.

1-((6-(8-((3,4-dimethoxyphenyl)amino)imidazo[1,2-a]pyrazin-6-yl)-1H-indol-3-yl)methyl)azetidin-3-ol S970.

Compound B (250 mg, 0.56 mmol) was dissolved in DCM (2.5 mL) and added trifluoroacetic acid (TFA) (2.5 mL) at 0° C. over a period of 10 minutes. The resulting solution was stirred at room temperature under N₂ atmosphere for 10 hours.

After completion of the reaction, the reaction mixture was concentrated to dry under reduced pressure and crude material was directly used for the next step without purification N-(3,4-dimethoxyphenyl)-6-(1H-indol-6-yl)imidazo[1,2-a]pyrazin-8-amine.

A solution of azetidin-3-ol (45 mg, 0.65 mmol) and 1,4 Dioxane (1 mL) was added dropwise to a stirred solution of formaldehyde (30% in H₂O, 83 μL) and acetic acid (1 mL) in 1,4 Dioxane (1 mL) at 0° C. over a period of 10 minutes. The resulting solution was stirred at same temperature for 30 min, A solution of crude N-(3,4-dimethoxyphenyl)-6-(1H-indol-6-yl)imidazo[1,2-a]pyrazin-8-amine in 1,4 Dioxane (1 mL) was added dropwise to the reaction mixture at 0° C. over a period of 10 minutes. The reaction mixture was allowed to room temperature and stirred at same temperature for 24 hours. The resulting suspension was concentrated under reduced pressure and the title product was isolated by normal phase silica gel chromatography (Dichloromethane: Methanol) to give an off-white solid (20 mg, 10%).

¹H NMR (500 MHz, DMSO) δ 11.11 (s, 1H), 9.47 (s, 1H), 8.56 (s, 1H), 8.12 (d, J=2.4 Hz, 1H), 8.04 (s, 1H), 8.00 (dd, J=10.1, 1.0 Hz, 1H), 7.67 (m, 1H), 7.63 (dd, J=5.6, 0.9 Hz, 3H), 7.27 (d, J=1.8 Hz, 1H), 6,98 (d, J=8.8 Hz, 1H), 4.18 (p, J=6.2 Hz, 1H), 3.83 (s, 3H), 3.77 (s, 3H), 3.74-3.69 (m, 2H), 3.48 (td, J=6.1, 1.9 Hz, 2H), 2.80 (td, J=6.3, 1.7 Hz, 2H)ppm; 13C NMR (126 MHz, DMSO) δ 173.1, 148.9, 145.1, 144.4, 138.1, 137.1, 134.5, 132.5, 132.4, 130.8, 127.7, 125.5, 119.3, 117.0, 116.7, 112.6, 111.8, 109.4, 107.9, 105.4, 63.9, 61.1, 56.3, 55.8, 54.4, 22.7; LCMS (ESI) m/z calcd for C₂₆H₂₇N₆O₃: 471.2139 Found: 471.2139 (M+1)+.

### Example 2

### In Vitro, Analysis of Syk Inhibitors

Syk kinase inhibition assays were run on 96-well plates using the ADP-Glo assay kit according to manufacturer's specifications. All kinase reactions were run using a 1× kinase reaction buffer consisting of 40 mM Tris pH 7.5, 20 mM MgCl₂, 0.1 mg/mL bovine serum albumin, and 0.1 mM dithriothreitol (DTT) as required by the kinase of interest (each of those listed in Table 2 above) to generate kinase reaction buffer. Stock solutions of kinase inhibitor in DMSO were diluted into 1× kinase reaction buffer and the final concentration of DMSO in each kinase reaction was held below 0.5%. The total volume of each kinase reaction was 25 μL apportioned as: 8.5 μL 1× kinase reaction buffer, 5 μL diluted kinase inhibitor, 5 μL 1 mg/mL Poly-(4:1-Glu, Tyr) kinase substrate peptide, 4 μL kinase in 1× kinase reaction buffer, and 2.5 μL 100 μM ATP diluted into kinase reaction buffer. Kinase inhibitors were incubated with kinase and substrate prior to addition of ATP for 30 minutes at ambient temperature.

After addition of ATP to the wells, the plate was incubated at 37° C. for 30 minutes. After incubation, 25 μL ADP-Glo reagent was added immediately and incubated at ambient temperature 30 minutes. After 30 minutes, 50 μL kinase detection reagent was added and the plate was incubated at ambient temperature for 45 minutes and protected from light. After 45 minutes, the luminescence was recorded using a Neo2 plate reader and 1 sec integration of the signal. The background signal was subtracted from all readings and the signal was normalized to the maximum signal obtained in the absence of inhibitor. Data were plotted and inhibition curves fit using Graphpad software and results are shown in Tables 2 and 3 above.

### Example 3

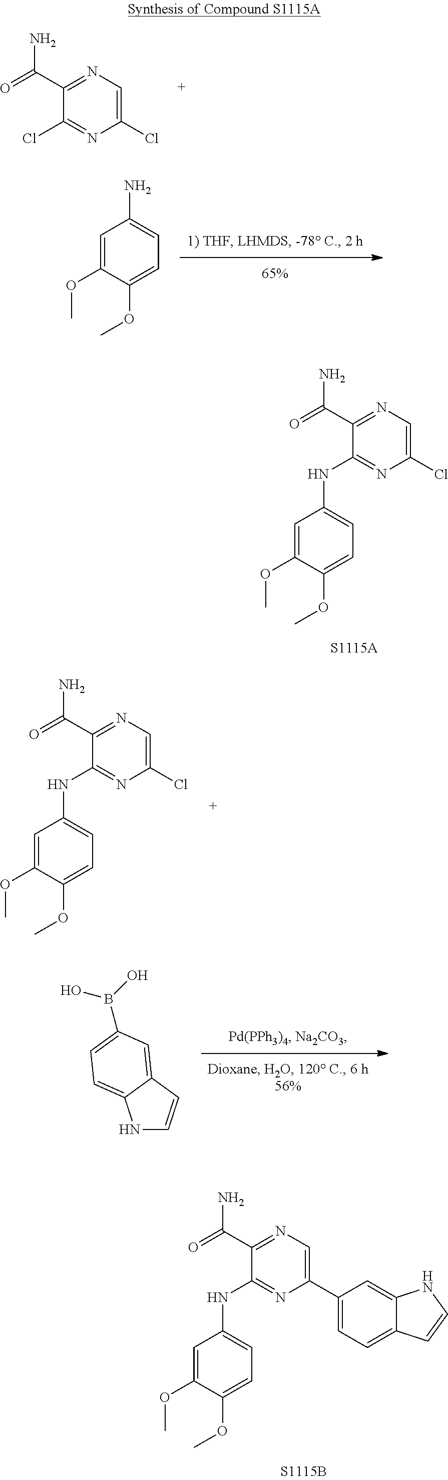 [SMILES: NC(=O)c1ncc(Cl)nc1Cl ; COc1ccc(N)cc1OC ; COc1ccc(Nc2nc(Cl)cnc2C(N)=O)cc1OC ; COc1ccc(Nc2nc(Cl)cnc2C(N)=O)cc1OC ; OB(O)c1ccc2[nH]ccc2c1 ; COc1ccc(Nc2nc(-c3ccc4cc[nH]c4c3)cnc2C(N)=O)cc1OC]

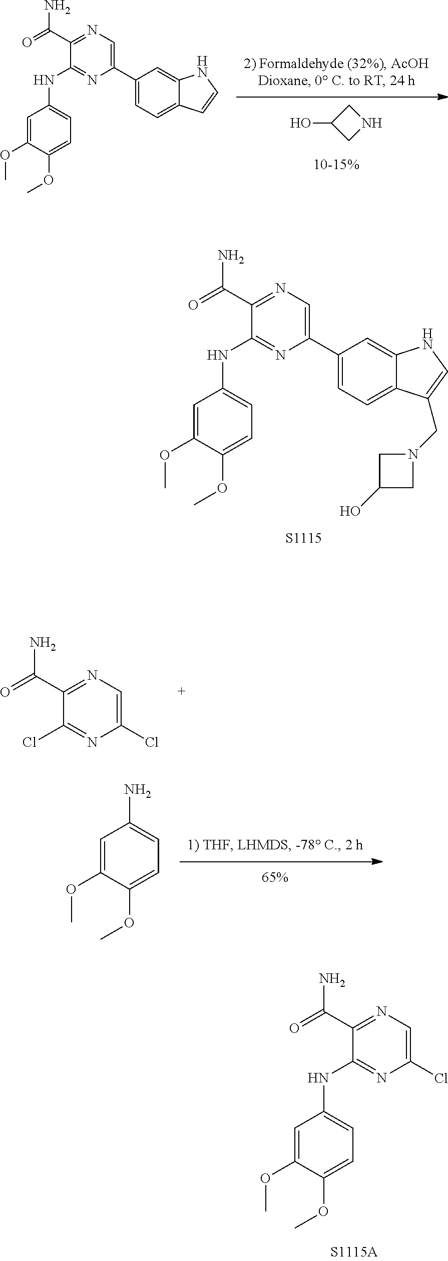 [SMILES: COc1ccc(Nc2nc(-c3ccc4cc[nH]c4c3)cnc2C(N)=O)cc1OC ; OC1CNC1 ; COc1ccc(Nc2nc(-c3ccc4c(CN5CC(O)C5)c[nH]c4c3)cnc2C(N)=O)cc1OC ; NC(=O)c1ncc(Cl)nc1Cl ; COc1ccc(N)cc1OC ; COc1ccc(Nc2nc(Cl)cnc2C(N)=O)cc1OC]

A solution of 3,5-dichloropyrazine-2-carboxamide (1.0 eq.) was added to a stirred solution of 3,4,-dimethyoxyaniline (1.0 eq.) in THF (20 V) and lithium bis(trimethylsilyl)amide (LHMDS) (3.0 eq.) at −78° C. The resulting reaction mixture was stirred at the same temperature for 2 hours then warmed to room temperature and stirred for 2 hours. After completion of the reaction (progress monitored by TLC), the reaction mixture was poured into ice-cold 2 N hydrochloric acid (HCl) and extracted with DCM and dried over sodium sulfate.

The solvents were then removed under vacuum. The crude intermediate obtained was triturated with hexanes to yield Compound S1115A (5-chloro-3-((3,4-dimehtoxyphenyl)amino)pyrazine-2-carboxamideas) in a dark yellow solid (Yield 70%).

### Example 4

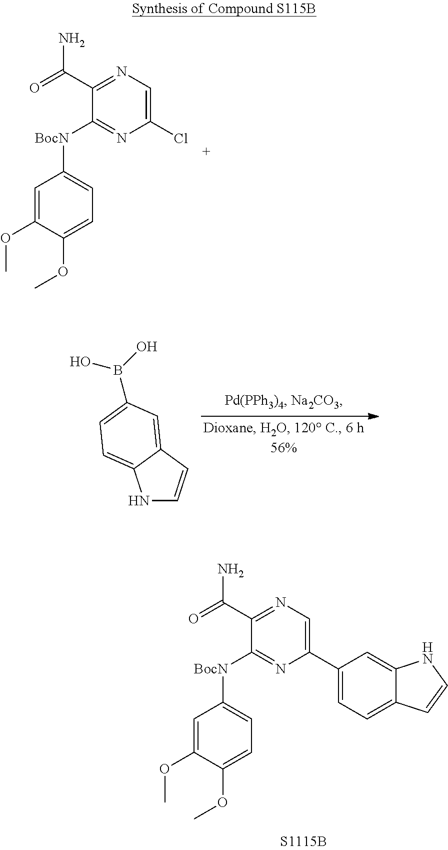 [SMILES: COc1ccc(N(C(=O)OC(C)(C)C)c2nc(Cl)cnc2C(N)=O)cc1OC ; OB(O)c1ccc2[nH]ccc2c1 ; COc1ccc(N(C(=O)OC(C)(C)C)c2nc(-c3ccc4cc[nH]c4c3)cnc2C(N)=O)cc1OC]

Compounds S1115-A (1.0 eq.) and (1H-indol-5-yl)boronic acid (1.2 eq.) were dissolved in 1,4 Dioxane (20 V) followed by the addition of H₂O (20 V) in a vial fitted with a screw cap having a septum. The reaction mixture was purged with a gentle stream of N₂ while stirring. After a 5-minute purge, the exit needle was removed and Pd (PPh₃)₄ (0.05 eq.) was added, followed quickly by Na₂CO₃(3.0 eq.).

The reaction mixture was heated to 120° C. and stirred at the same temperature for 6 hours. Then the reaction mixture was concentrated to dryness and the desired product was isolated by normal phase silica gel chromatography (DCM: MeOH) to result in Compound S1115B (3-((3,4-methoxyphenyl)amino)-5-(1H-indol-6-yl)pyrazine-2-carboxamide) as a yellow solid. (Yield 58%).

### Example 5

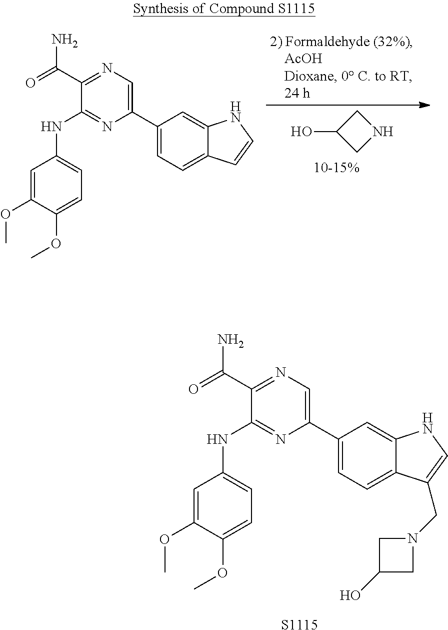 [SMILES: COc1ccc(Nc2nc(-c3ccc4cc[nH]c4c3)cnc2C(N)=O)cc1OC ; OC1CNC1 ; COc1ccc(Nc2nc(-c3ccc4c(CN5CC(O)C5)c[nH]c4c3)cnc2C(N)=O)cc1OC ; "S1115"]

A solution of azetidine-3-ol (3.0 eq.) and 1,4 Dioxane (20 V) was added dropwise to a stirred solution of formaldehyde (30% in H₂O, 3.0 eq.) and acetic acid (10 eq.) in 1,4 Dioxane (10 V) at 0° C. over a period of 10 minutes.

The resulting solution was stirred at the same temperature for 30 minutes, and a solution of Compound S1115B in 1,4 Dioxane (20 V) was added dropwise to the reaction mixture at 0° C. over a period of 10 minutes. The reaction mixture was allowed to reach room temperature and stirred at the same temperature for 24 hours. The resulting suspension was concentrated under reduced pressure and Compound S1115 (3-((3,4,-methoxyphenyl)amino)-5-(3-((3-hydroxyazetidin-1-yl)methyl)-1H-indol-6-yl)pyrazine-2-carboxamide) was isolated by reverse phase silica gel chromatography (pH 7.4, Ammonium acetate: CAN) to give Compound S 1115 in a yellow solid (10-15% yield).

1. A compound of formula X:
A-B-C(X)
or a pharmaceutically acceptable salt thereof, wherein:
A is a first ring that is nitrogen-linked and optionally substituted, the first ring comprising a fused bicyclic ring or a monocyclic ring with each ring each first ring comprising a 4-6-membered heterocycle or carbocycle;
B is a second ring, optionally substituted with at least one substituent, the second ring comprising a monocyclic ring or a fused bicyclic or tricyclic ring, and wherein each ring of the second ring comprises a 5- or 6-membered heterocycle or carbocycle; and
(is a third bicyclic ring comprising a 5- or 6-membered carbocycle fused with a 5- or 6-membered carbocycle or heterocycle, wherein the third bicyclic ring is substituted with at least a first substituent comprising: 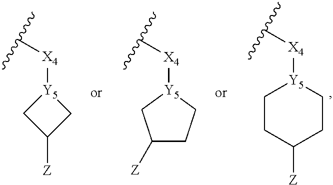
or an amine, or a ketone, or a carboxamide, or an alcohol,
wherein:
X₄ is CH₂, NH, CO, O, or S,
Y₅ is CH or N,
Z is selected from OH, SH, NH₂, CO₂H, NHCH₃, and 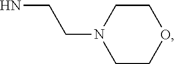
and [symbol]
is a point of attachment of the first substituent to the third bicyclic ring.

2. The compound of claim 1, wherein A is: 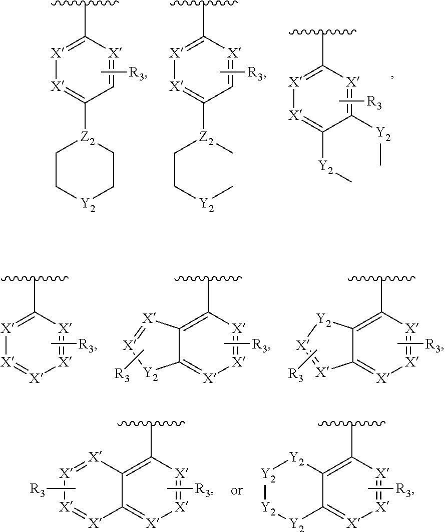
wherein:
each X′ is independently selected from CH and NH;
each Y₂ is independently selected from CH₂, NH, O, and S;
Z₂ is CH or N; and
each R₃ is independently selected from H, F, Cl, OH, and —Y—(CH₂)ₙ—, which n=1-14.

3. The compound of claim 1, wherein B is 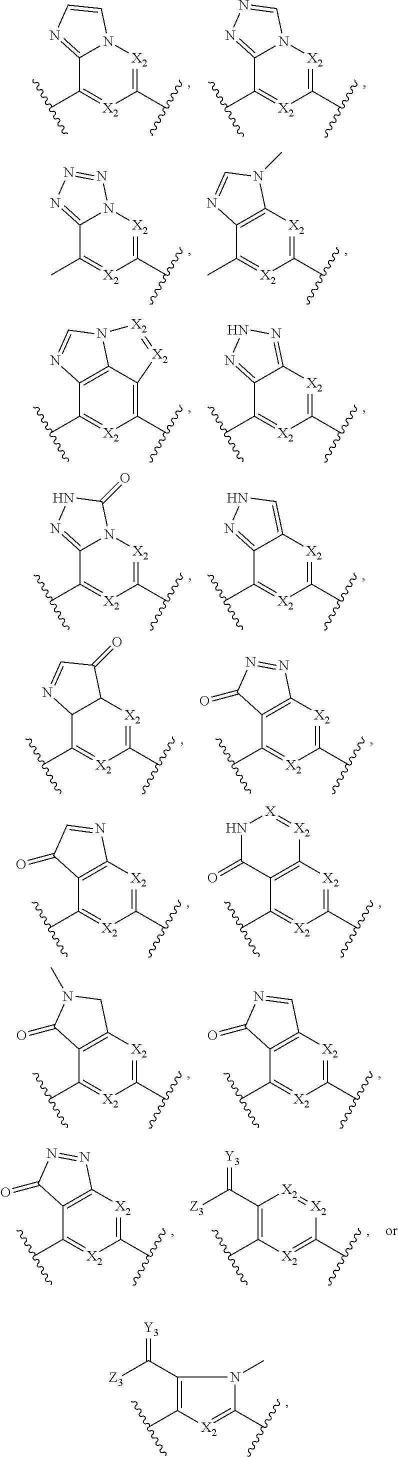
wherein:
each X₂ is independently selected from CH and N;
Y₃ is selected from O, NH and S; and
Z₃ is selected from OH, NH₂, NHCH₃.

4. The compound of any one of claims 1-3, with the proviso that B is not

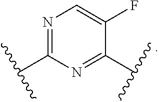 [SMILES: *c1ncc(F)c(*)n1]

5. The compound of any one of claims 1-3, wherein one or more hydrogens can be optionally substituted with deuterium.

6. The compound of claim 1, wherein the N linked to the first ring of A forms a nitrogen bridge with B.

7. The compound of claim 6, wherein A is a first monocyclic ring substituted with a bulky substituent para to the nitrogen bridge.

8. The compound of claim 7, wherein the bulky substituent is morpholine.

9. The compound of any one of claims 1-3 or 6-8, wherein each ring of the first ring of A is a 6-membered, optionally substituted, open or closed, heterocycle or carbocycle.

10. The compound of any one of claims 1-3 or 6-8, wherein A is a first monocyclic ring substituted with two methoxy groups.

11. The compound of any one of claims 1-3 or 6-8, wherein B is a monocyclic ring substituted with an amide.

12. The compound of any one of claims 1-3 or 6-8, wherein B is rigid.

13. The compound of claim 1, wherein B is a pyrazine substituted with at least one substituent.

14. The compound of claim 13, wherein the at least one substituent of B is an amide.

15. The compound of claim 1, wherein C is 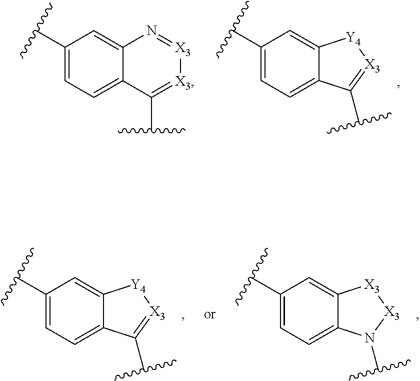
wherein:
each X₃ is independently selected from CH and N; and
Y₄ is CH₂, NH, O, or SH.

16. The compound of claim 15, wherein the first substituent of C is

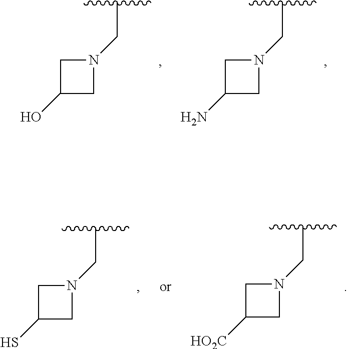 [SMILES: *CN1CC(O)C1 ; *CN1CC(N)C1 ; *CN1CC(S)C1 ; *CN1CC(C(=O)O)C1]

17. The compound of claim 1, wherein Z of the first substituent is —OR in which R is 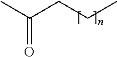
and n=1-14.

18. The compound of any one of claims 1-3, 6-8, or 13-17, wherein the third bicyclic ring of C is an indole.

19. The compound claim 1, wherein the third bicyclic ring of C is an indole and the first substituent of C comprises a 4-6-membered, optionally substituted, carbo- or heterocycle substituent.

20. The compound of claim 19, wherein the carbo- or heterocycle substituent comprises a lipophilic ester.

21. The compound of any one of claims 1-3, 6-8, or 13-17, wherein Z of the first substituent of C comprises a phosphonate or phosphate ester.

22. The compound of any one of claims 1-3, 6-8, or 13-17, wherein the first substituent of C is 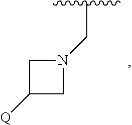
wherein Q is a phosphate or phosphonate ester.

23. The compound of any one of claims 1-3, 6-8, or 13-17, wherein the first substituent of C is 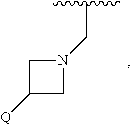
wherein Q is RC(═O)O—, in which R is an alkyl.

24. The compound of any one of claims 1-3, 6-8, or 13-17, wherein B can form a hydrogen bond with amino acid residue Ala451 in a catalytic domain of Syk tyrosine kinase and the first substituent of C can form a hydrogen bond with amino acid residue Asp512 in a catalytic domain of Syk tyrosine kinase.

25. The compound of claim 1 having a structure: 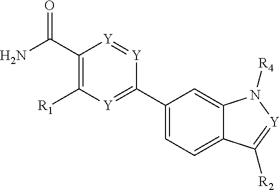
wherein:
R₁ is 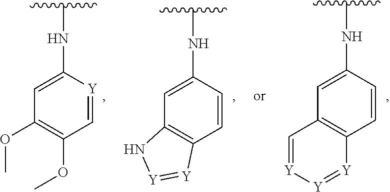
Y is each independently CH or N,
R₂ is 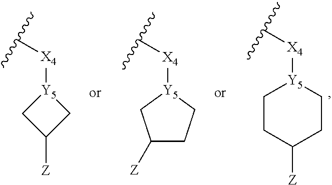
or an amine, or an alcohol, wherein:
X₄ is CH₂, NH, CO, O, or S,
Y₅ is CH or N,
Z is selected from OH, SH, NH₂, CO₂H, NHCH₃, and 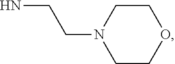
and [symbol]
is a point of attachment; and
R₄ is H, CH₃, an amine, or a methanol.

26. The compound of claim 1 having a structure: 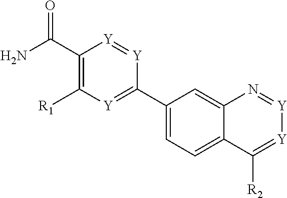
wherein:
R₁ is 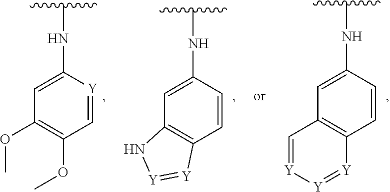
R₂ is 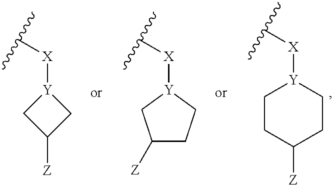
or an amine, or a propanol;
X is each independently CH₂, NH, CO, O, or S,
Y is each independently CH or N,
Z is selected from OH, SH, NH₂, CO₂H, NHCH₃, and 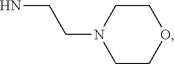
and [symbol]
is a point of attachment.

27. The compound of any one of claims 1-3, 6-8, 13-17, 19, 25, or 26, wherein the compound has:
a CLogP value between about 2 and about 5 (e.g., between 2 and about 5, about 2 and 5, or 2 and 5);
a polar surface area of less than about 250 square Å (e.g., less than 250 square Å);
no more than 5 H-bond donors;
no more than 15 H-bond acceptors; and/or
no more than 20 rotatable bonds.

28. The compound of any one of claims 1-3, 6-8, 13-17, 19, 25, or 26, wherein the compound binds to a Syk tyrosine kinase with specificity.

29. The compound of any one of claims 1-3, 6-8, 13-17, 19, 25, or 26, wherein, when administered to a subject, the compound inhibits Syk from phosphorylating an erythrocyte anion transporter Band 3.

30. The compound of claim 1 having a structure: 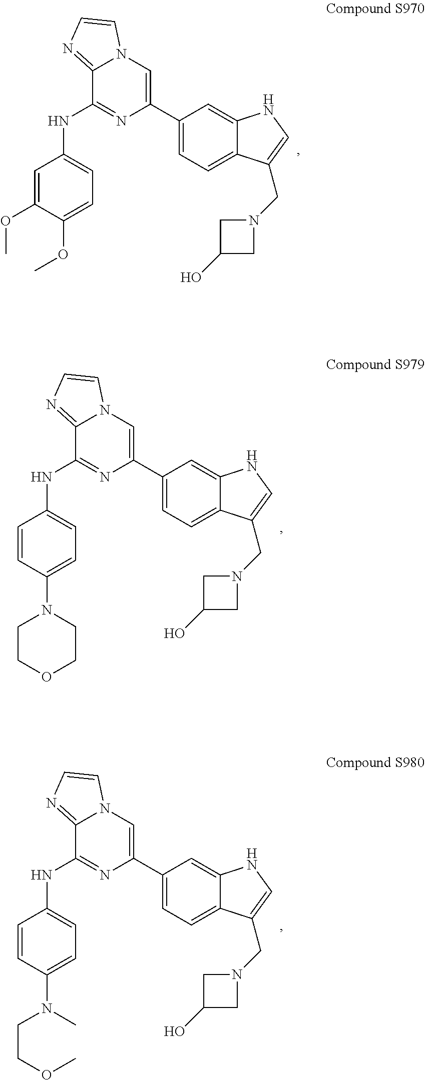 [SMILES: COc1ccc(Nc2nc(-c3ccc4c(CN5CC(O)C5)c[nH]c4c3)cn3ccnc23)cc1OC ; OC1CN(Cc2c[nH]c3cc(-c4cn5ccnc5c(Nc5ccc(N6CCOCC6)cc5)n4)ccc23)C1 ; COCCN(C)c1ccc(Nc2nc(-c3ccc4c(CN5CC(O)C5)c[nH]c4c3)cn3ccnc23)cc1] 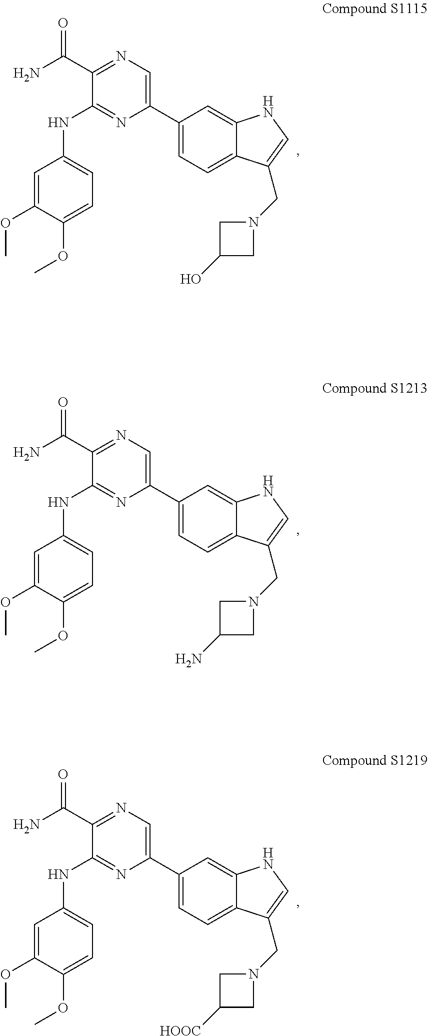 [SMILES: COc1ccc(Nc2nc(-c3ccc4c(CN5CC(O)C5)c[nH]c4c3)cnc2C(N)=O)cc1OC ; COc1ccc(Nc2nc(-c3ccc4c(CN5CC(N)C5)c[nH]c4c3)cnc2C(N)=O)cc1OC ; COc1ccc(Nc2nc(-c3ccc4c(CN5CC(C(=O)O)C5)c[nH]c4c3)cnc2C(N)=O)cc1OC] 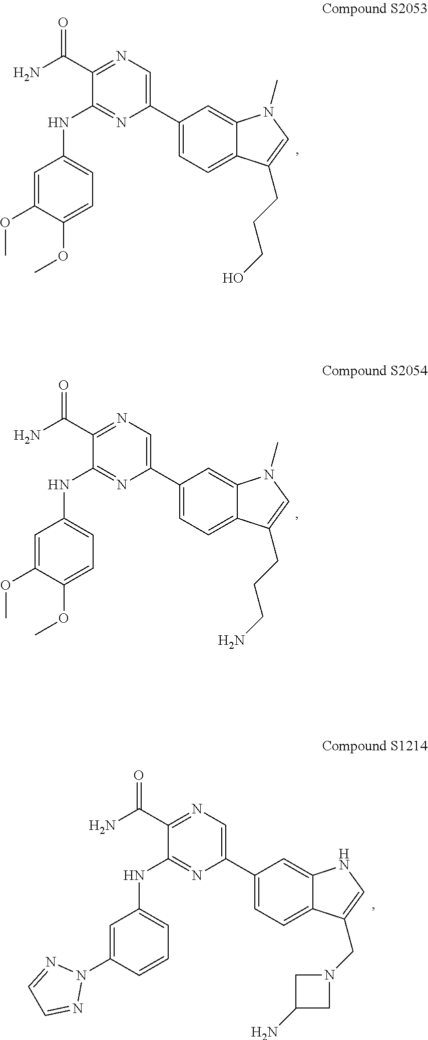 [SMILES: COc1ccc(Nc2nc(-c3ccc4c(CCCO)cn(C)c4c3)cnc2C(N)=O)cc1OC ; COc1ccc(Nc2nc(-c3ccc4c(CCCN)cn(C)c4c3)cnc2C(N)=O)cc1OC ; NC(=O)c1ncc(-c2ccc3c(CN4CC(N)C4)c[nH]c3c2)nc1Nc1cccc(-n2nccn2)c1] 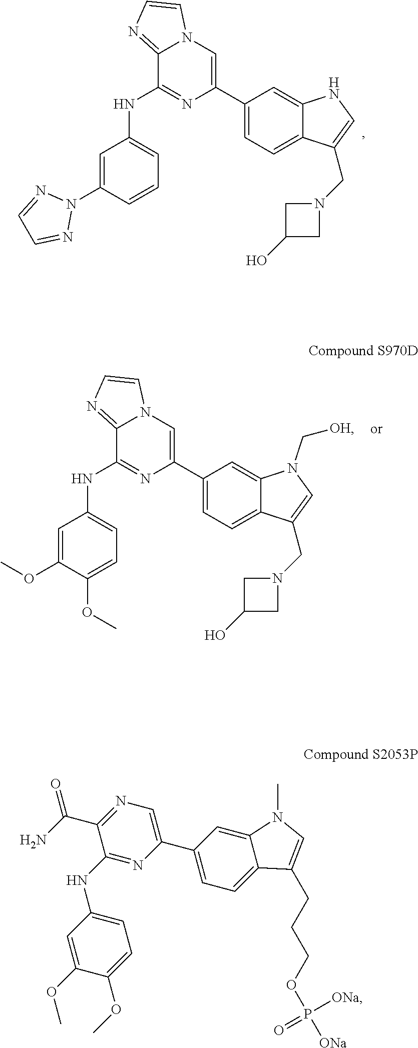
or a pharmaceutically acceptable salt of any of the foregoing, structures.

31. The compound of claim 1 having a structure: 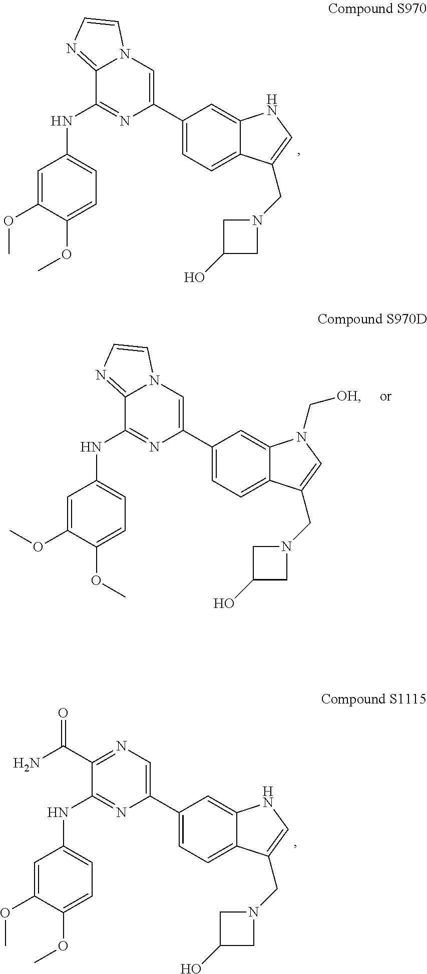 [SMILES: COc1ccc(Nc2nc(-c3ccc4c(CN5CC(O)C5)c[nH]c4c3)cn3ccnc23)cc1OC ; COc1ccc(Nc2nc(-c3ccc4c(CN5CC(O)C5)cn(CO)c4c3)cn3ccnc23)cc1OC ; COc1ccc(Nc2nc(-c3ccc4c(CN5CC(O)C5)c[nH]c4c3)cnc2C(N)=O)cc1OC]
or a pharmaceutically acceptable salt of any of the foregoing structures.

31. The compound of claim 1 having a structure: 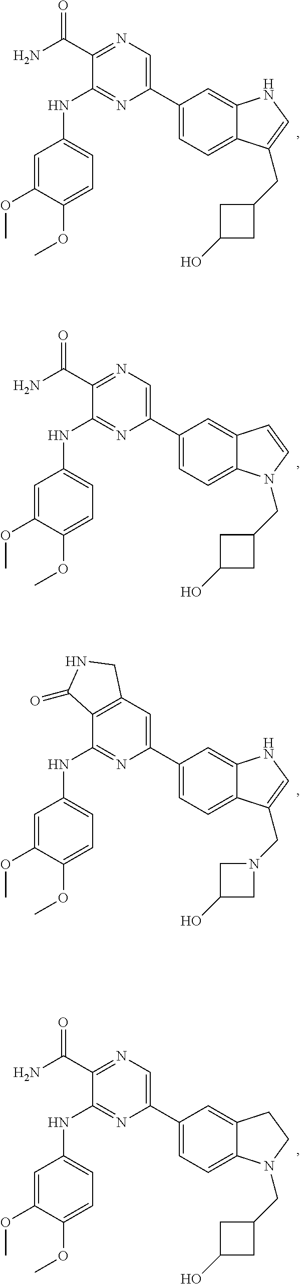 [SMILES: COc1ccc(Nc2nc(-c3ccc4c(CC5CC(O)C5)c[nH]c4c3)cnc2C(N)=O)cc1OC ; COc1ccc(Nc2nc(-c3ccc4c(ccn4CC4CC(O)C4)c3)cnc2C(N)=O)cc1OC ; COc1ccc(Nc2nc(-c3ccc4c(CN5CC(O)C5)c[nH]c4c3)cc3c2C(=O)NC3)cc1OC ; COc1ccc(Nc2nc(-c3ccc4c(c3)CCN4CC3CC(O)C3)cnc2C(N)=O)cc1OC] 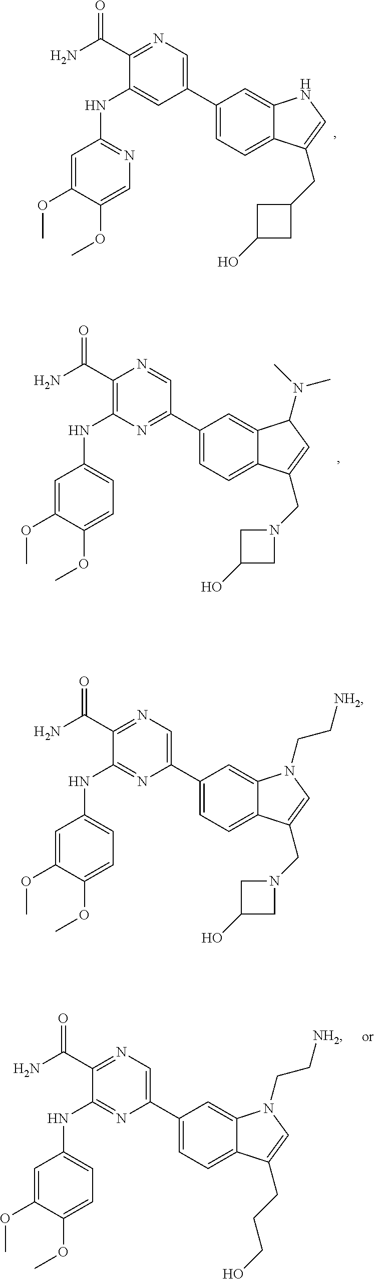 [SMILES: COc1cnc(Nc2cc(-c3ccc4c(CC5CC(O)C5)c[nH]c4c3)cnc2C(N)=O)cc1OC ; COc1ccc(Nc2nc(-c3ccc4c(c3)C(N(C)C)C=C4CN3CC(O)C3)cnc2C(N)=O)cc1OC ; COc1ccc(Nc2nc(-c3ccc4c(CN5CC(O)C5)cn(CCN)c4c3)cnc2C(N)=O)cc1OC ; COc1ccc(Nc2nc(-c3ccc4c(CCCO)cn(CCN)c4c3)cnc2C(N)=O)cc1OC] 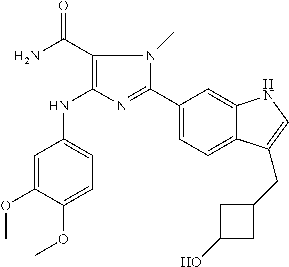 [SMILES: COc1ccc(Nc2nc(-c3ccc4c(CC5CC(O)C5)c[nH]c4c3)n(C)c2C(N)=O)cc1OC]
or a pharmaceutically acceptable salt of any of the foregoing structures.

32. The compound of claim 31, wherein the hydroxyl (HO—) group of the compound is replaced with a phosphate or a phosphonate ester.

33. The compound of claim 31, wherein the hydroxyl (HO—) group of the compound is replaced with RC(═O)O—, in which R is an alkyl.

34. The compound of claim 33, wherein the alkyl is a C₁-C₆ alkyl.

35. A pharmaceutical composition comprising a compound of any one of claims 1-34 and a pharmaceutically acceptable carrier or excipient.

36. The pharmaceutical composition of claim 35, further comprising one or more of cremophor, polysorbate, and nanoparticles.

37. The pharmaceutical composition of claim 35, further comprising a polymer or a hydrogel.

38. The Method of treating a red blood cell (RBC)-mediated disease, the method comprising administering to a subject an effective amount of:
a compound or pharmaceutically acceptable salt of any one of claims 1-34, or
a pharmaceutical composition of any one of claims 35-37.

39. The method of claim 38, further comprising administering to the subject an effective amount of at least one second active agent.

40. A method of treating an RBC-mediated disease, the method comprising administering to a subject an effective amount of:
a compound of formula X:
A-B-C (X)
or a pharmaceutically acceptable salt thereof, wherein:
A is a first ring that is nitrogen-linked and optionally substituted, the first ring comprising a fused bicyclic ring or a monocyclic ring with each ring each first ring comprising a 4-6-membered heterocycle or carbocycle;
B is a second ring, optionally substituted with at least one substituent, the second ring comprising a monocyclic ring or a fused bicyclic or tricyclic ring, and wherein each ring of the second ring comprises a 5- or 6-membered heterocycle or carbocycle; and
C is a third bicyclic ring comprising a 5- or 6-membered carbocycle fused with a 5- or 6-membered carbocycle or heterocycle, wherein the third bicyclic ring is substituted with at least a first substituent comprising: 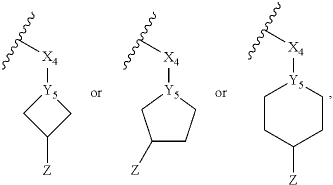
or an amine, or a ketone, or a carboxamide, or an alcohol,
wherein:
X₄ is each independently CH₂, NH, CO, O, or S,
Y₅ is each independently CH or N,
Z is selected from OH, SH, NH₂, CO₂H, NHCH₃, and 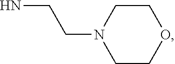
and [symbol]
is a point of attachment of the first substituent to the third bicyclic ring,
wherein the compound inhibits Syk tyrosine kinase at a concentration that is at least 20-fold lower than the concentration at which the compound inhibits HGFR, EGFR, FGFR3, IGF1R, and/or a VEGFR; or
a pharmaceutical composition comprising the compound or a pharmaceutically acceptable salt thereof.

41. The method of any one of claims 35-40, wherein the RBC-mediated disease is selected from the group consisting of sickle cell disease, thalassemia, glucose-6-phosphate dehydrogenase deficiency, glutathione reductase deficiency, and a disease involving the release of microvesicles from blood cells.

42. The method of any one of claims 38-40, wherein the RBC-mediated disease is α-thalassemia or β-thalassemia, alone or in combination with sickle cell disease.

43. A method of preventing or inhibiting the phosphorylation of a human erythrocyte anion transporter band 3 in a subject comprising administering to the subject an effective amount of:
a compound or pharmaceutically acceptable salt of any one of claims 1-36, or
a pharmaceutical composition of any one of claims 35-37.

44. The method of any one of claims 38-43, wherein the compound or pharmaceutical composition stabilizes erythrocyte cell membranes in the subject and reduces the release of hemoglobin from sickle cells in the subject.

45. The method of any one of claims 38-44, wherein the risk of vaso-occlusive crisis in the subject is reduced.

46. The method of any one of claims 38-45, wherein administration of the compound, pharmaceutical salt thereof, or pharmaceutical composition inhibits Syk tyrosine kinase at an IC₅₀ of 200 nM or lower.

47. The method of any one of claims 38-45, wherein the subject is a human child.

48. The method of any one of claims 38-45, which further comprises the simultaneous or sequential administration, in either order, of an effective amount of at least one second active agent.

49. The method of claim 48, wherein the at least one second active agent is selected from the group consisting of fostamatinib, PRT062607, TAK-659, TAE-684, entospletinib, lanraplenib, cerdulatinib, piceatannol, S701, Syk II, Syk IV, TA805567, GSK143, Syk-IN-3, Syk-IN-4, SRX3207, R09021, gusacitinib, R112, PRT-060318, and OXSI-2, optionally wherein the at least one active agent is formulated as a pharmaceutical composition comprising a pharmaceutically acceptable carrier or excipient.

50. The method of claim 48, wherein the compound, pharmaceutically acceptable salt thereof, or pharmaceutical composition and the at least one second agent are administered by the same route.

51. The method of claim 48, wherein the compound, pharmaceutically acceptable salt thereof, or pharmaceutical composition and the at least one second agent are administered by different routes.

52. The method of claim 48, wherein the at least one second active agent comprises hydroxyurea.

53. The method of claim 49, wherein the at least one second active agent comprises more than one second active agent which are all selected from the group consisting of fostamatinib, PRT062607, TAK-659, TAE-684, entospletinib, lanraplenib, cerdulatinib, piceatannol, S701, Syk II, Syk IV, TAS05567, GSK143, Syk-IN-3, Syk-IN-4, SRX3207, R09021, gusacitinib, R112, PRT-060318, and OXSI-2, optionally wherein the at least one active agent is formulated as a pharmaceutical composition comprising a pharmaceutically acceptable carrier or excipient.

54. A method of treating a subject for a disease involving the release of microvesicles from blood cells, which method comprises administering to the subject an effective amount of a compound or pharmaceutically acceptable salt thereof, the compound comprising a rigid, fused bicyclic ring, which is substituted with at least two substituents, at least one of which is an N-linked, optionally substituted, carbo- or heterocycle and the other of which is an indole substituted with at least one substituent comprising a 4-6-membered, optionally substituted, carbo- or heterocycle, whereupon the subject is treated for the disease involving the release of microvesicles.

55. The method of claim 54, wherein the disease is α-thalassemia or β-thalassemia, alone or in combination with sickle cell disease.

56. The method of claim 54, wherein the disease is glucose-6-phosphate dehydrogenase (G6PD) deficiency or glutathione reductase deficiency.

57. The method of any one of claims 38-47, which further comprises the simultaneous or sequential administration, in either order of, a Src inhibitor, optionally as a pharmaceutical composition comprising a pharmaceutically acceptable carrier or excipient.

58. The method of claim 57, wherein the compound, pharmaceutically acceptable salt thereof, or pharmaceutical composition and the Src inhibitor are administered by the same or different routes.

59. The method of claim 57 or 58, wherein the Src inhibitor is dasatinib, ibrutinib, bafetinib, PP1, PP2, PP121, or a combination of two or more of the foregoing.


SEQUENCE LISTING (1 sequence)

SEQ ID NO: 1 - protein, 635 aa, Homo sapiens
  source 1..635: note = spleen tyrosine kinase (Syk) protein
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN